(19) United States
(12) Patent Application Publication
(10) Pub. No.: US 2005/0026826 A1
(43) Pub. Date: Feb. 3, 2005

(54) Feline proinsulin, insulin and constituent peptides

(76) Inventor: Margarethe Hoenig, Bogart, GA (US)

Correspondence Address: MUETING, RAASCH & GEBHARDT, P.A., P.O. BOX 581415, MINNEAPOLIS, MN, 55458, US

(21) Appl. No.: 10/760,928

(22) Filed: Jan. 20, 2004

Related U.S. Application Data
(60) Provisional application No. 60/444,009, filed on Jan. 31, 2003.
(60) Provisional application No. 60/440,964, filed on Jan. 17, 2003.

(51) Int. Cl. A61K 38/17; C07K 7/08

(52) U.S. Cl. 514/12; 530/324

(57) ABSTRACT

The amino acid sequences of feline proinsulin and structurally related polypeptides such as insulin and the A, B and C chains are provided. Also provided are peptidomimetics, analogs, polypeptide subunits, polynucleotides that encode the polypeptides and subunits thereof, methods of making the polypeptides and polynucleotides, antibodies, peptide aptamers, and diagnostic and therapeutic methods.

### CROSS-REFERENCE TO RELATED APPLICATIONS

This application claims the benefit of U.S. Provisional Application Ser. No. 60/440,964, filed 17 Jan. 2003; and U.S. Provisional Application Ser. No. 60/444,009, filed 31 Jan. 2003, which are incorporated herein by reference in their entirety.

### BACKGROUND OF THE INVENTION

Diabetes is a relatively common endocrinopathy in the cat. The incidence is approximately 0.5-1%. Several risk factors have been identified: age, obesity, neutering and gender (Panciera et al., Am. Vet. Med. Assoc. 197, 1504-1508 (1990); Scarlett et al., JAVMA 212, 1725-1731 (1998)). Over 50% of diabetic cats were over 10 years old and age was identified as the most important single risk factor. Obesity is thought to increase the risk of developing diabetes 3- to 5-fold. Neutered cats have nearly twice the risk and male cats 1.5 times the risk of developing diabetes. Diabetes in young cats is extremely rare (Woods et al., J. Am. Anim. Hosp. Assoc. 30, 177-180 (1994); Root et al., J. Small Anim. Pract. 36, 416-420 (1995)).

It is thought that diabetic cats have primarily type 2 diabetes, based on the fact that most diabetic cats have islet amyloid (Yano et al., Vet. Pathol. 18, 621-627 (1981)) which has been called the hallmark of type 2 diabetes (Westermark et al., Diabetologia 15, 417-421 (1978)). However, although most cats have changes similar to type 2 diabetes, they do not respond well to oral antidiabetic agents. For example, less than 25% of diabetic cats are reported to respond to the oral drug glipizide. Moreover, glipizide has been found to lead to amyloidosis in cats (Hoenig et al., Am. J. Pathol. 157, 2143-2150 (2000)). In that study, only 1 of the cats that were treated with insulin showed minor amyloid deposition whereas amyloid was seen in all of the glipizide treated cats. It is possible that cats are not diagnosed early in the disease process and consequently already have marked structural beta cell impairment at the time of diagnosis. Treatment with insulin would therefore be expected to yield better results, since insulin allows the beta cells to rest and regenerate. However, it has been observed that treatment of diabetic cats with available human recombinant and animal source (PZI insulin, which is 90% beef and 10% pork insulin, Idexx Laboratories, Westbrook, Me.) insulin preparations often does not lead to the desired response. Feline insulin would provide the veterinarian with another option for control of blood glucose concentrations and is especially useful because it is the native species insulin.

In mammalian cells, insulin is formed from the cleavage of proinsulin. The naturally occurring proinsulin molecule contains, from the N-terminus to the C-terminus, the B chain, the C chain, and the A chain. The B chain and the C chain are connected by a dipeptide linkage (Arg-Arg) that is cleaved during intracellular processing. The C chain and the A chain are likewise connected by a dipeptide linkage (Lys-Arg) that is also cleaved during intracellular processing. Cleavage of proinsulin yields an insulin molecule and a C-peptide molecule.

Early detection and treatment of diabetes is essential to halt progression of the disease. Specific insulin/proinsulin assays are required for the early detection of beta cell dysfunction. Proinsulin measurements have recently become available in human medicine, enabling the routine measurement of this ratio. In people, the earliest marker is a change in insulin/proinsulin secretion and a change in the insulin/proinsulin ratio. Proinsulin is sorted from other beta cell-derived polypeptides during its passage through the Golgi and is transported to secretory vesicles where it is converted to insulin and C-peptide by two distinct, Ca-requiring endopeptidases (Halban, Diabetologia 37 [Suppl.2]: S65-72 (1994)). Insulin and C-peptide are formed in equimolar ratio. Proinsulin and proinsulin-like peptides have been shown to have pathophysiologic significance in humans and serve as the earliest indicator of beta cell dysfunction. It has become clear that elevated fasting proinsulin levels are early indicators of even minor beta cell damage, regardless of whether diabetes develops later (for review see Halban, Diabetologia 37 [Suppl.2]: S65-72 (1994)).

Species-specific assays for proinsulin, insulin, and C-peptide exist for several animals. However, there are no proinsulin, insulin or C-peptide assays that use feline proinsulin or constituents thereof as standards. In other words, early changes in beta cell function in cats cannot be readily detected because feline-specific reactants are not available. Insulin assays performed by veterinary laboratories therefore must resort to using insulin from other species (such as pork, human recombinant and beef) as standards. Depending on the standard and assay procedure used, the insulin concentration in a given sample may be interpreted as high or low by different laboratories. This has made it difficult, if not impossible, to reliably diagnose beta cell secretory changes in feline diabetes.

Elucidation of species-specific proinsulin, insulin and C-peptide is thus very important for the detection of beta cell failure. To have such an early marker of beta cell dysfunction is particularly important in the cat where no other early indicators of impending beta cell failure have been identified. A C-peptide assay would additionally allow the detection of endogenous insulin secretion in cats receiving insulin therapy. Further, since insulin therapy is the preferred treatment for diabetic cats, the production of recombinant feline insulin is especially desirable because of the lack of availability of cat pancreata for extraction of the naturally occurring hormone as has been done for years to obtain pork and beef insulin. Just as the recombinant production of human recombinant insulin was a major advance in the treatment of human diabetics, so is the discovery and characterization of feline insulin and related molecules a major therapeutic and diagnostic advance in veterinary medicine.

### SUMMARY OF THE INVENTION

The invention provides feline proinsulin, insulin, subunits of feline proinsulin, and constituent peptides of proinsulin. The feline proinsulin can have an amino acid sequence that is at least 85% identical to SEQ ID NO:1. The feline proinsulin can also have an amino acid sequence that is at least single unit percentages greater than 85% identical to SEQ ID NO:1, for example 86%, 87%, and 88% identity, and so on. Preferably, the feline proinsulin has an amino acid sequence that is at least 90% identical to SEQ ID NO:1. More preferably, the feline proinsulin has an amino acid sequence that is at least 95% identical to SEQ ID NO:1. Most preferably, the feline proinsulin has an amino acid sequence that is identical to SEQ ID NO:1.

The constituent peptides of proinsulin include the B-chain peptide, the A-chain peptide, and the C-chain peptide. The feline C-chain peptide can have an amino acid sequence that is at least 80% identical to SEQ ID NO:9. The feline C-chain peptide can also have an amino acid sequence that is at least single unit percentages greater than 80% identical to SEQ ID NO:9, for example 81%, 82%, and 83% identity, and so on. Preferably, the feline C-chain peptide has an amino acid sequence that is at least 90% identical to SEQ ID NO:9. More preferably, the feline C-chain peptide has an amino acid sequence that is at least 95% identical to SEQ ID NO:9. Even more preferably, the feline C-chain peptide has an amino acid sequence that is at least 99% identical to SEQ ID NO:9. Most preferably, the feline C-chain peptide has an amino acid sequence that is identical to SEQ ID NO:9.

The subunits of feline proinsulin are preferably at least five amino acids in length, more preferably the subunits are at least seven amino acids in length, even more preferably the subunits are at least ten amino acids in length, and most preferably the subunits are at least twelve amino acids in length. Preferably the subunits of feline proinsulin are biologically active.

The invention also provides analogs and peptidomimetics of feline proinsulin, insulin, subunits of feline proinsulin, and constituent peptides of proinsulin and insulin. Preferably the analogs and petidomimetics are biologically active. The analogs and petidomimetics may contain one or more amino acid substitutions or derivatizations.

Polynucleotides that encode feline proinsulin, insulin, subunits of feline proinsulin, and constituent peptides of proinsulin are also provided. In a preferred embodiment, these polynucleotides are inserted into, and form part of, a vector. The polynucleotides of the invention can be inserted into an expression cassette or an expression vector. The polynucleotides can be, for example, ribonucleic acid, deoxyribonucleic acid, or derivatives or analogs thereof.

The invention provides antibodies and peptide aptamers that bind to feline proinsulin, insulin, subunits of feline proinsulin, and/or constituent peptides of proinsulin. Preferably the antibody or peptide aptamer is specific for the feline molecule and does not bind to human, porcine, or bovine proinsulin, insulin, A-chain peptide, B-chain peptide, and/or C-chain peptide. Preferably the antibody or peptide aptamer binds to only one of the following feline peptides: A-chain peptide, B-chain peptide, C-chain peptide, insulin, and/or proinsulin. The antibody can be a polyclonal antibody or a monoclonal antibody.

The invention provides a pharmaceutical composition containing a pharmaceutically acceptable carrier and a peptide, polypeptide, subunit, analog, peptidomimetic, antibody, or peptide aptamer as described herein. In a preferred embodiment, the pharmaceutical composition is formulated for transdermal administration, oral administration, intravenous administration, intraocular administration, intranasal administration, inhalation administration, parenteral administration, and/or rectal administration.

The invention further provides methods for making feline proinsulin, insulin, subunits thereof, and their constituent peptides. In one embodiment, feline proinsulin, insulin, subunits thereof, and their constituent peptides are isolated from tissue. In another embodiment, feline proinsulin, insulin, subunits thereof, and their constituent peptides are synthesized using genetic engineering. In yet another embodiment, feline proinsulin, insulin, subunits thereof, and their constituent peptides are synthesized using chemical methods. Preferably feline insulin is made by cleaving a proinsulin molecule to yield C-peptide and insulin. Alternatively, A-peptide and B-peptide can be separately synthesized and combined to form insulin.

A method for diagnosing diabetes in a cat is also provided by the invention. The method involves detecting feline proinsulin, insulin, and/or one or more of their constituent peptides in a biological fluid obtained from a cat. Examples of biological fluids than can be used include blood, serum, plasma, or urine. The method involves determining the ratio of insulin to proinsulin, or determining the ratio of insulin to C-peptide. A bioassay may be used to quantify the amount of insulin, proinsulin, and/or C-peptide. Alternatively, chromatography may be used to quantify the amount of insulin, proinsulin, and/or C-peptide. Chemiluminescent methods using horseradish peroxidase or alkaline phosphatase and bead separation may also be used to quantify the amount of insulin, proinsulin, and/or C-peptide. Western blot analysis can also be used, as can a competitive or noncompetitive immunoassay. The immunoassay can be, for example, an immunoenzymometric assay, enzymoimmunassay, an immunofluorometric assay, or a radioimmunoassay. Peptide aptamers or antibodies that bind specifically to feline proinsulin, insulin, and/or one or more of its constituent peptides can be used in the diagnostic method.

The invention further provides a method to determine if a cat is predisposed to develop neuropathy, retinopathy, or nephropathy. The method involves determining if the C-peptide concentration in a biological fluid obtained from the cat is less than a predetermined value. In humans, this level is about 0.7 ng/ml.

A method for treating diabetes in a cat is also provided. The treatment method of the invention involves administering feline insulin, proinsulin, C-peptide, a subunit, analog, or peptidomimetic thereof to a cat suspected of having or known to have diabetes. Preferably the feline insulin, proinsulin, C-peptide, a subunit, analog, or peptidomimetic is formulated as a pharmaceutical composition.

The invention provides a method to identify an antiproliferative factor. In one embodiment, the method involves incubating neuroblastoma test cells with feline C-peptide, insulin, and a candidate antiproliferative factor; and comparing proliferation of the test cells to proliferation of neuroblastoma control cells that were incubated with feline C-peptide and insulin. In another embodiment, the method involves incubating neuroblastoma test cells with feline C-peptide, insulin, and a candidate antiproliferative factor; and comparing autophosphorylation of insulin receptors within the test cells to autophosphorylation of insulin receptors within control cells that were incubated with feline C-peptide and insulin.

The invention further provides a method to reduce or ameliorate a diabetes-associated disorder in a mammal. The method involves administering an effective amount of a feline C-peptide, a peptidomimetic of feline C-peptide, a subunit of a feline C-peptide, an analog, a peptidomimetic of a subunit of a feline C-peptide, or any combination thereof to the mammal in need of such treatment.

Kits are also provided by the invention. A kit can contain packaging material and an antibody that specifically binds to the feline C-peptide. Preferably a kit contains packaging material and a first antibody that specifically binds to the feline C-peptide portion of feline proinsulin, and a second antibody that specifically binds to feline insulin. More preferably a kit contains packaging material and a first antibody that specifically binds to a feline C-peptide, and a second antibody that specifically binds to feline insulin. Preferably the first antibody or the second antibody is coupled to a detectable marker. More preferably the first antibody and the second antibody are each bound to a detectable marker.

### BRIEF DESCRIPTION OF THE DRAWINGS

FIG. 1 shows an amino acid alignment of feline (86 amino acids, SEQ ID NO:1), human (86 amino acids, SEQ ID NO:2), porcine (84 amino acids, SEQ ID NO:3) and bovine (81 amino acids, SEQ ID NO:4) proinsulin. The B chain includes residues 1-30 (feline, SEQ ID NO:5; human, SEQ ID NO:6; porcine, SEQ ID NO:7; bovine, SEQ ID NO:8); the C chain include residues 33-63 (feline, SEQ ID NO:9; human, SEQ ID NO:10; porcine, SEQ ID NO:11; bovine SEQ ID NO:12); and the A chain includes residues 66-86 (feline, SEQ ID NO:13; human, SEQ ID NO:14; porcine, SEQ ID NO:15; bovine SEQ ID NO:16). Dipeptide linkages (Arg3 1 -Arg32 and Lys64-Arg-65) separate the C chain from the B chain and the A chain, respectively.

FIG. 2 shows selected analogs of human insulin. C 14-FA, myristoylic acid (Nature Rev. Drug Discovery 1:529-540 (2002)).

FIG. 3 shows the naturally occurring nucleotide sequence encoding feline proinsulin (SEQ ID NO:22) and the sequence optimized for expression in E. coli (SEQ ID NO:33)

### DETAILED DESCRIPTION OF ILLUSTRATIVE EMBODIMENTS

Feline Proinsulin, Insulin, Constituent Polypeptides and Peptidomimetics

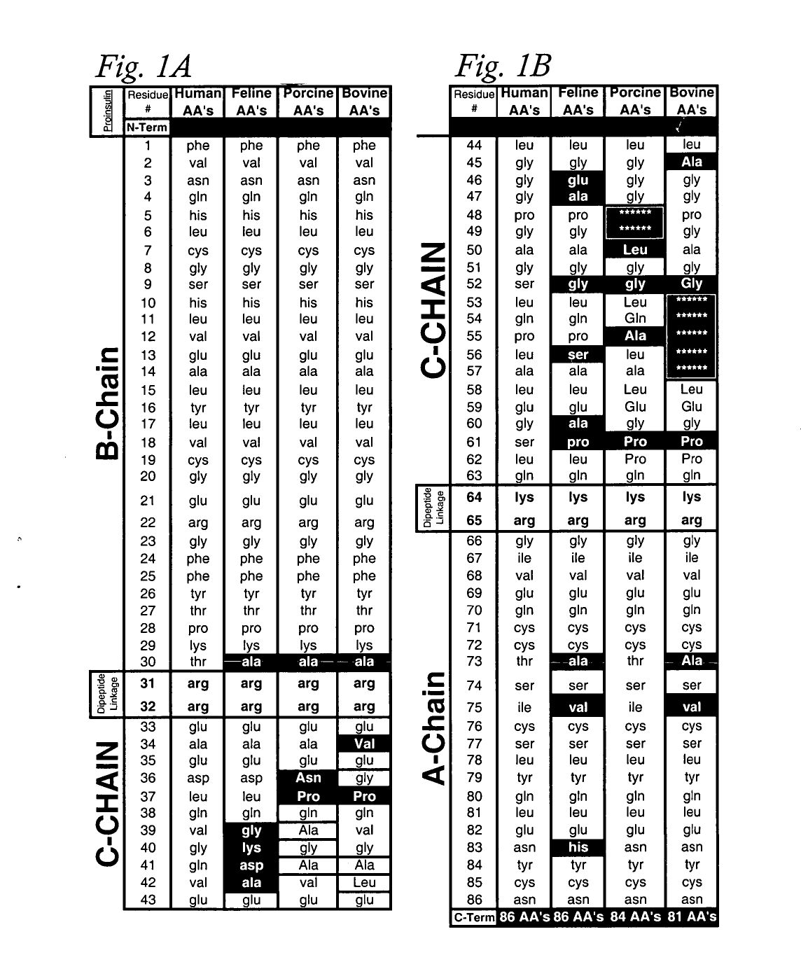

The invention provides a novel feline proinsulin polypeptide (SEQ ID NO:1). As in other mammalian systems, proinsulin in the cat includes constituent peptides known as the A-, B- and C-chains (SEQ ID NOs:13, 5 and 9, respectively) (FIG. 1). As indicated in FIG. 1, the amino-terminal to carboxyl-terminal order of the feline proinsulin chain is as follows, B-chain (amino acids 1-30), Arg-Arg linker (amino acids 31-32), C-chain (amino acids 33-63), Lys-Arg linker (amino acids 64-65), and the A-chain (amino acids 66-86). Proteolytic processing of proinsulin yields the A-, B-, and C-chains. The A- and B-chains (SEQ ID NOs: 13 and 5) then link together via disulfide bond formation to form feline insulin. The C-chain (SEQ ID NO:9), when so liberated, is often referred to as “C-peptide” or “C-chain peptide” and is included in the invention. The mature C-peptide does not include the Arg-Arg linker (amino acids 31-32), or the Lys-Arg linker (amino acids 64-65) sequences of the feline proinsulin.

The invention also includes constituent peptides of feline proinsulin that include the proteolytically cleaved products of proinsulin. Constituent peptides include B-chain peptides that include none, one or both arginine residues of the Arg-Arg linker (amino acids 31-32). Constituent peptides include C-peptides that include none, one or both arginine residues of the Arg-Arg linker (amino acids 31-32); none, one or both of the arginine or lysine residues of the Arg-Lys linker (amino acids 64-65); or one or both arginine residues of the Arg-Arg linker (amino acids 31-32), and one or both of the arginine or lysine residues of the Arg-Lys linker (amino acids 64-65). Additional examples of constituent peptides include A-chain peptides that include none, one or both of the arginine or lysine residues of the Arg-Lys linker (amino acids 64-65).

Biologically active analogs and subunits of feline proinsulin, insulin and the individual A-, B- and C-chains are included in the invention as well. Such subunits are exemplified by those having the amino acid sequence E L G E A P G A G (SEQ ID NO:34), or E A P L Q (SEQ ID NO:35). These subunits are expected to stimulate Na(+),K(+)-ATPase activity (see, e.g., U.S. Pat. No. 6,610,649). Polynucleotides encoding feline proinsulin, insulin and their constituent peptides, as well as biologically active analogs and subunits thereof, are also encompassed by the invention.

As used herein, the term “polypeptide” refers broadly to a polymer of two or more amino acids joined together by peptide bonds. The term “polypeptide” also includes molecules such as insulin which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

A “biologically active” feline proinsulin or insulin analog, subunit or derivative is a polypeptide that is able to decrease blood glucose concentrations. One bioassay for insulin measures the increase of glycogen of isolated rat diaphragm in glucose medium and glucose uptake by the rat epididymal fat pad (L. Vu et al., Anal. Biochem.,1998, 262: 17-22; A. Christopher et al., Indian J. Med. Res., 1974, 62: 1499-1510; A. Moody, Experientia, 1964, 20: 646-648; and K. Gundersen et al., Diabetes 1965, 14: 805-810). Another bioassay constitutes as radioreceptor bioassay which is based on the competition of labeled and unlabeled hormone for binding to a specific tissue receptor (such as rat erythrocyte membrane, fat or liver cells) (K. Gambhir et al., Biochem. Med. Metab., Biol. 1991, 45: 133-153; M. Laburthe et al., Diabetologia, 1975, 11: 517-526). Insulin preparations used to treat humans often contain not less than 27.5 USP Insulin Human Units per mg of insulin on the dried basis (USP DI 1997; 17^{th} edition). A standard insulin preparation for administration to humans is a mixture of 52% bovine and 48% porcine insulin containing 24 units per mg. Three international units of human insulin are contained in approximately 130 microgram (with sucrose 5 mg) in one ampule of the first International Reference Preparation for Immunoassay (1974). See Martindale “The ExtraPharmacopeia”, page 844, 28^{th} edition; editor: J. Reynolds, The Pharmaceutical Press, 1982, citing Bangham et al.; J. Biol. Stand., 1978, 6, 301).

A biologically active peptide of the invention may improve renal function in diabetics when administered with insulin, beyond the effect of insulin treatment alone. The C-peptide is an example of a biologically active peptide having this activity. C-peptide also has cardioprotective effects and increases blood flow, probably through an effect on nitric oxide (L. Young et al., Am. J. Physiol. Heart Circ Physiol. 2000; 279: H1453-1459; B. Johansson et al., Diabet Med 2000; 17: 181-189). The C-peptide can also reduce duration-dependent hippocampal apoptosis resulting from type-I diabetes, promote neurite proliferation, promote neurite outgrowth, promote autophosphorylation of an insulin receptor, stimulate phosphoinositide 3-kinase, stimulate p38 mitogen-activated protein kinase, promote expression of nuclear factor-kappaB, promotes nuclear tranlocation of nuclear factor-kappaB, promote expression of Bcl2, stimulate Na(+),K(+)-ATPase activity, stimulate nitric oxide synthase activity, raise intracellular Ca⁺² concentration, reduce nerve dysfunction in patients with diabetic neuropathy, and/or reduce c-jun N-terminal kinase phosphorylation in diabetic mammals when administered with insulin. A biologically active peptide may exhibit one or more than one of the above described activities. These activities may be assayed in a variety of cell types. For example, neuroblastoma cells are preferred cells in which to assay for the biological activity of a C-peptide.

A biologically active “subunit” of a feline proinsulin or insulin includes a feline proinsulin or insulin that has been truncated at either the N-terminus, or the C-terminus, or both, by one or more amino acids, as long as the truncated polypeptide retains bioactivity and contains at least 5 amino acids, more preferably at least 7 amino acids, even more preferably at least 10 amino acids, and most preferably at least 12 amino acids.

A biologically active “analog” of a feline proinsulin or insulin includes a feline proinsulin that has been modified by the addition, substitution, or deletion of one or more contiguous or noncontiguous amino acids, or that has been chemically or enzymatically modified, e.g., by attachment of a reporter group, by an N-terminal, C-terminal or other functional group modification or derivatization, or by cyclization, as long as the analog retains biological activity. An analog can thus include additional amino acids at one or both of the termini of a polypeptide.

Substitutes for an amino acid in the polypeptides of the invention are preferably conservative substitutions, which are selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Examples of preferred conservative substitutions include Lys for Arg and vice versa to maintain a positive charge; Glu for Asp and vice versa to maintain a negative charge; Ser for Thr so that a free —OH is maintained; and Gln for Asn to maintain a free NH₂.

Other amino acids and derivatives thereof that can be used include 3-hydroxyproline, 4-hydroxyproline, homocysteine, 2-aminoadipic acid, 2-aminopimelic acid, γ-carboxyglutamic acid, β-carboxyaspartic acid, omithine, homoarginine, N-methyl lysine, dimethyl lysine, trimethyl lysine, 2,3-diaminopropionic acid, 2,4-diaminobutyric acid, homoarginine, sarcosine, hydroxylysine, substituted phenylalanines, norleucine, norvaline, 2-aminooctanoic acid, 2-aminoheptanoic acid, statine, β-valine, naphthylalanines, substituted phenylalanines, tetrahydroisoquinoline-3-carboxylic acid, and halogenated tyrosines.

Preferred biologically active analogs of feline proinsulin or any of its constituent peptides include those analogs that are at least 85% identical, more preferably at least 90% identical, even more preferably at least 95% identical, and most preferably at least 98% identical to feline proinsulin or its constituent peptides. Preferred biologically active analogs of feline insulin (comprising A and B chains) include those analogs that 85% identical, more preferably at least 90% identical, even more preferably at least 95% identical, and most preferably at least 98% identical to feline insulin, provided that the A-chain of the feline insulin analog contains a histidine at amino acid position 83 relative to feline proinsulin (FIG. 1). Such analogs contain one or more amino acid deletions, insertions, and/or substitutions relative to feline proinsulin or insulin, and may further include chemical and/or enzymatic modifications and/or derivatizations as described above.

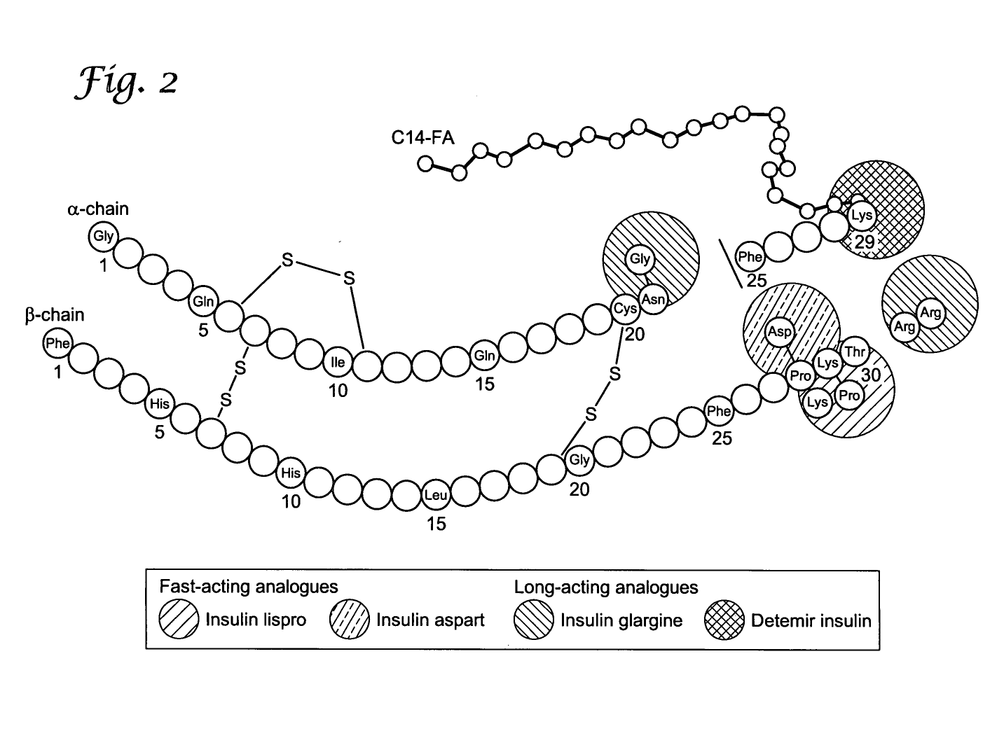

Particularly preferred analogs of feline insulin include those that are structurally analogous to human insulin analogs now on the market, including insulin lispro, insulin aspart, insulin glargine and detemir insulin (FIG. 2). These insulin analogs have modified amino acid sequences and improved pharmacokinetic properties. For example, the absorption of insulin after subcutaneous injection can be improved by increasing the rate of dissociation of insulin molecules into monomers. Insulin lispro and insulin aspart are rapid-acting analogs that have reduced self-association as a result of protein engineering. In insulin lispro, a lysine-proline (Lys-Pro) sequence at the end of the insulin-B chain is reversed, which creates steric hindrance and a reduced ability to self-associate. Insulin aspart incorporates an amino-acid change (Pro B28 to aspartic acid (Asp)) that also creates charge repulsion and steric hindrance due to a local conformational change at the carboxyl terminus of the B chain. Both are absorbed more rapidly than regular insulin and reduce post-prandial glucose excursions more efficiently. Because of their short-lived action, adjustments in basal insulin levels are required to achieve improvements in overall glycemic control.

Long-acting insulin analogs to fulfill basal insulin requirements have been produced, either by introducing amino-acid changes that increase the isoelectric point of insulin and reduce its solubility at physiological pH, or by covalent acylation. These longer-acting analogs are preferred in the treatment of feline diabetes because they would allow less frequent (e.g., once daily) administration of insulin. Insulin glargine is a long-acting insulin that contains two extra arginine molecules at the end of the B chain (Arg B31 and Arg B32) to alter the isoelectric point. A glycine substitution at A21 (A chain) was made to stabilize the molecule. After subcutaneous injection in human subjects, insulin levels rise slowly to a plateau within 6-8 hours and remain essentially unchanged for up to 24 hours, suitable for once-daily administration. Acylation of the amino group of Lys B29, as in insulin detemir (N-myristoyl des (B30) human insulin), promotes reversible binding of insulin to albumin, thereby delaying its absorption from the subcutaneous tissue and transport across the capillary endothelium of skeletal muscle. Nature Rev. Drug Discovery 1:529-540 (2002).

The invention also provides polyproteins. Generally, polyproteins include two or more polypeptides of the invention that are continuously linked into a single amino acid chain. The polypeptides can be connected by linkers (see U.S. Pat. No. 6,558,924). Such a polyprotein can be isolated and then cleaved to produce polypeptides or coupled polypeptides of the invention. The polyprotein can be cleaved through use of numerous methods, such as chemical or protease cleavage. Accordingly, linkers can be designed to be cleaved by specific proteases or chemicals. Examples of chemicals that can be used to cleave polyproteins of the invention include cyanogen bromide (-Met↓-), formic acid (70%) and heat (-Asp↓Pro-), hydroxylamine at pH 9 and heat (Asn↓Gly-), iodosobenzoic acid-2-(2-nitrophenyl)-3-methyl-3-bromoindole-nine in 50% acetic acid (-Trp↓), and the like. Examples of enzymes that can be used to cleave polyproteins of the invention include Ala-64 subtilisin (-Gly-Ala-His-Arg↓), clostripain (-Arg↓ and Lys-Arg↓), collagenase (-Pro-Val↓Gly-Pro-), enterokinase (-Asp-Asp-Asp-Asp-Lys↓), factor Xa (-Ile-Glu (or Asp)-Gly-Arg↓), renin (-Pro-Phe-His-Leu↓Leu-), a-thrombin (-Leu-Val-Pro-Arg↓Gly-Ser-), trypsin (-Arg↓ or -Lys↓), chymotrypsin, tobacco etch virus protease (-Glu-Asn-Leu-Tyr-Phe-Gln↓Gly-), and the like. Polyproteins may be used to increase the production efficiency of the peptides of the invention. Peptides are often times difficult to produce due to proteolytic susceptibility and inefficient expression. These difficulties may be overcome through use of polyproteins due to increased protease resistance and expression. Methods to produce polyproteins are known in the art (U.S. Pat. No. 6,127,150).

The invention provides fusion polypeptides having a carrier polypeptide coupled to a polypeptide of the invention. A carrier polypeptide may be used to increase or decrease the solubility of a fusion polypeptide. The carrier polypeptide may also be used to increase the immunogenicity of the fusion polypeptide to increase production of antibodies that bind to a polypeptide of the invention. The invention is not limited by the types of carrier polypeptides used to create fusion polypeptides of the invention. Examples of carrier polypeptides include, keyhole limpet hemacyanin, bovine serum albumin, ovalbumin, mouse serum albumin, rabbit serum albumin, and the like. The carrier polypeptides may also be used to provide for the separation or detection of a fusion polypeptide. Accordingly, a fusion polypeptide can be detected or isolated by interaction with other components that bind to the carrier polypeptide portion of the fusion polypeptide. For example, a fusion polypeptide having avidin as a carrier polypeptide can be detected or separated with biotin through use of known methods. A carrier polypeptide may also be used to cause the fusion polypeptide to form an inclusion body upon expression within a cell. A carrier polypeptide can also be an export signal that causes export of a fusion polypeptide out of a cell, or directs a fusion polypeptide to a compartment within a cell, such as the periplasm.

For example, an expression cassette can be designed to express a polyprotein that includes biotin coupled to ten copies of a polypeptide of the invention that are connected to each other by a chemical or protease cleavable linker. The polyprotein can be expressed within a cell and then bound to an avidin support such that the polyprotein is immobilized. Cellular contaminants can then be washed away to allow isolation of the polyprotein. The polyprotein can then be cleaved to release polypeptides of the invention. These peptides can be purified through use of numerous art recognized methods, such as gel filtration chromatography, ion exchange chromatography, and the like.

A carrier polypeptide may be coupled to polypeptide of the invention through use of routine recombinant methods. A carrier polypeptide may also be coupled to a polypeptide of the invention through use of chemical linking methods, or through use of a chemical linker. Such coupling methods are known in the art and have been described. Harlow et al., Antibodies: A Laboratory Manual, page 319 (Cold Spring Harbor Pub. 1988); Taylor, Protein Immobilization, Marcel Dekker, Inc., New York, (1991).

The invention provides peptidomimetics of the polypeptides of the invention. A peptidomimetic describes a peptide analog, such as those commonly used in the pharmaceutical industry as non-peptide drugs, with properties analogous to those of the template polypeptide. (Fauchere, J., Adv. Drug Res., 15: 29 (1986), Evans et al., J. Med. Chem., 30:1229 (1987), and U.S. Pat. No. 6,664,372). Peptidomimetics are structurally similar to polypeptides having peptide bonds, but have one or more peptide linkages optionally replaced by a linkage such as, —CH₂NH—, —CH₂S—, —CH₂—CH₂—, —CH═CH— (cis and trans), —COCH₂—, —CH(OH)CH₂—, and —CH₂SO—, by methods known in the art. Advantages of peptidomimetics over natural polypeptide embodiments may include more economical production, greater chemical stability, altered specificity and enhanced pharmacological properties such as half-life, absorption, potency and efficacy.

Substitution of one or more amino acids within polypeptide or polypeptide mimetic with a D-amino acid of the same type (e.g., D-lysine in place of L-lysine) may be used to generate polypeptides and peptide mimetics that are more stable and more resistant to endogenous proteases.

Polypeptides, analogs, subunits, fusion polypeptides, and peptidomimetics of the invention can be modified for in vivo use by the addition, at the amino-terminus and/or the carboxyl-terminus, of a blocking agent to decrease degradation in vivo. This can be useful in those situations in which the polypeptide termini tend to be degraded by proteases in vivo. Such blocking agents can include, without limitation, additional related or unrelated peptide sequences that can be attached to the amino and/or carboxyl terminal residues of the polypeptide, subunit, analog, fusion polypeptide, and peptidomimetic to be administered. This can be done during chemical synthesis, or by recombinant DNA technology by methods familiar to artisans of average skill. Alternatively, blocking agents such as pyroglutamic acid, or other molecules known in the art, can be attached to the amino and/or carboxyl terminal residues, or the amino group at the amino terminus or carboxyl group at the carboxyl terminus can be replaced with a different moiety. Accordingly, the invention provides polypeptides, analogs, and peptidomimetics that are amino-terminally and carboxyl-terminally blocked.

Polypeptides, polyproteins, and fusion polypeptides of the invention can be produced on a small or large scale through use of numerous expression systems that include, but are not limited to, cells or microorganisms that are transformed with a recombinant vector into which a polynucleotide of the invention has been inserted. Such recombinant vectors and methods for their use are described below. These vectors can be used to transform a variety of organisms. Examples of such organisms include bacteria (for example, E. coli or B. subtilis); yeast (for example, Saccharomyces and Pichia); insects (for example, baculovirus); plants; or mammalian cells (for example, COS, CHO, BHK, 293, VERO, HeLa, MDCK, W138, and NIH 3T3 cells). Also useful as host cells are primary or secondary cells obtained directly from a mammal that are transfected with a vector.

Synthetic methods may also be used to produce polypeptides and polypeptide subunits of the invention. Such methods are known and have been reported (Merrifield, Science, 85:2149 (1963), U.S. Pat. Nos. 5,595,887; 5,116,750; 5,168,049 and 5,053,133; Olson et al., Peptides, 9, 301, 307 (1988)). The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). Polypeptides can be readily purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography, and the like. Polypeptides can also be readily purified through binding of a fusion polypeptide to separation media, followed by cleavage of the fusion polypeptide to release a purified polypeptide. For example, a fusion polypeptide that includes a factor Xa cleavage site between the polypeptide and the carrier polypeptide can be created. The fusion polypeptide can be bound to an affinity column to which the carrier polypeptide portion of the fusion polypeptide binds. The fusion polypeptide can then be cleaved with factor Xa to release the polypeptide. Such a system has been used in conjunction with a factor Xa removal kit for purification of the polypeptides of the invention.

Polynucleotides, Nucleic Acid Constructs, and Expression Cassettes

The invention provides polynucleotides that encode the polypeptides of the invention. The term “polynucleotide” refers broadly to a polymer of two or more nucleotides covalently linked in a 5′ to 3′ orientation. The terms nucleic acid, nucleic acid molecule, and oligonucleotide and protein included within the definition of polynucleotide and these terms are used interchangeably. It should be understood that these terms do not connote a specific length of a polymer of nucleotides, nor are they intended to imply or distinguish whether the polynucleotide is produced using recombinant techniques, chemical or enzymatic synthesis, or is naturally occurring.

Polynucleotides can be single-stranded or double-stranded, and the sequence of the second, complementary strand is dictated by the sequence of the first strand. The term “polynucleotide” is therefore to be broadly interpreted as encompassing a single stranded nucleic acid polymer, its complement, and the duplex formed thereby. “Complementarity” of polynucleotides refers to the ability of two single-stranded polynucleotides to base pair with each other, in which an adenine on one polynucleotide will base pair with a thymidine (or uracil, in the case of RNA) on the other, and a cytidine on one polynucleotide will base pair with a guanine on the other. Two polynucleotides are complementary to each other when a nucleotide sequence in one polynucleotide can base pair with a nucleotide sequence in a second polynucleotide. For instance, 5′-ATGC and 5′-GCAT are fully complementary, as are 5′-GCTA and 5′-TAGC.

Preferred polynucleotides of the invention include polynucleotides having a nucleotide sequence that is “substantially complementary” to (a) a nucleotide sequence that encodes a novel feline proinsulin polypeptide according to the invention, or (b) the complement of such nucleotide sequence. “Substantially complementary” polynucleotides can include at least one base pair mismatch, such that at least one nucleotide present on a second polynucleotide, however the two polynucleotides will still have the capacity to hybridize. For instance, the middle nucleotide of each of the two DNA molecules 5′-AGCAAATAT and 5′-ATATATGCT will not base pair, but these two polynucleotides are nonetheless substantially complementary as defined herein. Two polynucleotides are substantially complementary if they hybridize under hybridization conditions exemplified by 2×SSC (SSC: 150 mM NaCl, 15 mM trisodium citrate, pH 7.6) at 55° C. Substantially complementary polynucleotides for purposes of the present invention preferably share at least one region of at least 20 nucleotides in length which shared region has at least 60% nucleotide identity, preferably at least 80% nucleotide identity, more preferably at least 90% nucleotide identity and most preferably at least 95% nucleotide identity. Particularly preferred substantially complementary polynucleotides share a plurality of such regions.

Percent identity between two polypeptide or polynucleotide sequences is generally determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by Tatusova et al. (FEMS Microbiol. Lett., 174, 247-250 (1999)), and available on the world wide web at www.ncbi.nlm.nih.gov/gorf/b12.html. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.”

Likewise, two nucleotide sequences are preferably compared using the Blastn program, version 2.0.11, of the BLAST 2 search algorithm, also as described by Tatusova et al. (FEMS Microbiol. Lett, 174, 247-250 (1999)), and available on the world wide web at www.ncbi.nlm.nih.govlblast.html. Preferably, the default values for all BLAST 2 search parameters are used, including reward for match=1, penalty for mismatch=-2, open gap penalty=5, extension gap penalty=2, gap x_dropoff=50, expect=10, wordsize=11, and filter on. Locations and levels of nucleotide sequence identity between two nucleotide sequences can also be readily determined using CLUSTALW multiple sequence alignment software (J. Thompson et al., Nucl. Acids Res., 22:4673-4680 (1994)), available at from the world wide web at www.ebi.ac.uk/clustalw/.

It should be understood that a polynucleotide that encodes a feline preproinsulin (see GenBank Accession number AB043535), proinsulin, insulin or constituent polypeptide according to the invention is not limited to a polynucleotide that contains all or a portion of naturally occurring genomic or cDNA nucleotide sequence, but also includes the class of polynucleotides that encode such polypeptides as a result of the degeneracy of the genetic code. For example, the naturally occurring nucleotide sequence SEQ ID NO:22 is but one member of the class of nucleotide sequences that encodes a polypeptide having amino acid SEQ ID NO:1 (the feline proinsulin amino acid sequence). The class of nucleotide sequences that encode a selected polypeptide sequence is large but finite, and the nucleotide sequence of each member of the class can be readily determined by one skilled in the art by reference to the standard genetic code, wherein different nucleotide triplets (codons) are known to encode the same amino acid. Likewise, a polynucleotide of the invention that encodes a biologically active analog or subunit of a feline proinsulin polypeptide includes the multiple members of the class of polynucleotides that encode the selected polypeptide sequence.

A polynucleotide that “encodes” a polypeptide of the invention optionally includes both coding and noncoding regions, and it should therefore be understood that, unless expressly stated to the contrary, a polynucleotide that “encodes” a polypeptide is not structurally limited to nucleotide sequences that encode a polypeptide but can include other nucleotide sequences outside (i.e., 5′ or 3′ to) the coding region.

The polynucleotides of the invention can be DNA, RNA, or a combination thereof, and can include any combination of naturally occurring, chemically modified or enzymatically modified nucleotides. As noted above, the polynucleotide can be equivalent to the polynucleotide encoding a feline proinsulin, insulin, or constituent polypeptide, or it can include said polynucleotide in addition to one or more additional nucleotides.

A polynucleotide of the invention may be inserted into a vector. A vector may include, but is not limited to, any plasmid, phagemid, F-factor, virus, cosmid, or phage. The vector may be in a double or single stranded linear or circular form. The vector can also transform a prokaryotic or eukaryotic host either by integration into the cellular genome or exist extrachromosomally (e.g. autonomous replicating plasmid with an origin of replication). The polynucleotide in the vector can be under the control of, and operably linked to, an appropriate promoter or other regulatory sequence for transcription in vitro or in a host cell, such as a eukaryotic cell, or a microbe, e.g. bacteria. A regulatory sequence refers to nucleotide sequences located upstream (5′ non-coding sequences), within, or downstream (3′ non-coding sequences) of a coding sequence, and which influence the transcription, RNA processing or stability, or translation of the associated coding sequence. Examples of regulatory sequences include enhancers, promoters, translation leader sequences, introns, and polyadenylation signal sequences. They include natural and synthetic sequences as well as sequences that may be a combination of synthetic and natural sequences. Regulatory sequences are not limited to promoters. However, some suitable regulatory sequences useful in the present invention will include, but are not limited to, constitutive promoters, tissue-specific promoters, development-specific promoters, inducible promoters and viral promoters.

The vector may be a shuttle vector that functions in multiple hosts. The vector may also be a cloning vector which typically contain one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech). A vector may be an expression vector that contains regulatory sequences which direct the expression of a polynucleotide that is inserted into the expression vector. Numerous vectors are commercially available and are known in the art (Stratagene, La Jolla, Calif.; New England Biolabs, Beverly, Mass.).

Methods to introduce a polynucleotide into a vector are well known in the art (Sambrook et al., Molecular Cloning: A Laboratory Manual, 3rd edition, Cold Spring Harbor Press, Cold Spring Harbor, N.Y. (2001)). Briefly, a vector into which a polynucleotide is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a polynucleotide into the vector. The polynucleotide that is to be inserted into the vector is treated with one or more restriction enzymes to create a linearized segment having a blunt end, a “sticky” end with a 5′ or a 3′ overhang, or any combination of the above. The polynucleotide may also be treated with a restriction enzyme and subsequently treated with another DNA modifying enzyme. Such DNA modifying enzymes include, but are not limited to, polymerase, exonuclease, phosphatase or a kinase, to create a polynucleotide that has characteristics useful for ligation of a polynucleotide into the vector.

The treated vector and polynucleotide are then ligated together to form a construct containing a polynucleotide according to methods known in the art (Sambrook et al., Molecular Cloning: A Laboratory Manual, 3rd edition, Cold Spring Harbor Press, Cold Spring Harbor, N.Y. (2001)). Briefly, the treated nucleic acid fragment and the treated vector are combined in the presence of a suitable buffer and ligase. The mixture is then incubated under appropriate conditions to allow the ligase to ligate the nucleic acid fragment into the vector.

The invention also provides an expression cassette which contains a regulatory sequence capable of directing expression of a particular polynucleotide of the invention, such as SEQ ID NO:21, either in vitro or in a host cell. The expression cassette is an isolatable unit such that the expression cassette may be in linear form and functional in in vitro transcription and translation assays. The materials and procedures to conduct these assays are commercially available from Promega Corp. (Madison, Wis.). For example, an in vitro transcript may be produced by placing a polynucleotide under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a polynucleotide.

Such an expression cassette may contain one or a plurality of restriction sites allowing for placement of the polynucleotide under the regulation of a regulatory sequence. The expression cassette can also contain a termination signal operably linked to the polynucleotide as well as regulatory sequences required for proper translation of the polynucleotide. The expression cassette containing the polynucleotide may be chimeric, meaning that at least one of its components is heterologous with respect to at least one of its other components. The expression cassette may also be one which is naturally occurring but has been obtained in a recombinant form useful for heterologous expression. Expression of the polynucleotide in the expression cassette may be under the control of a constitutive promoter or an inducible promoter that initiates transcription only when the host cell is exposed to some particular external stimulus.

The expression cassette may include in the 5′-3′ direction of transcription, a transcriptional and translational initiation region, a polynucleotide and a transcriptional and translational termination region functional in vivo and /or in vitro. The termination region may be native with the transcriptional initiation region, may be native with the polynucleotide, or may be derived from another source.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA- box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

The invention also provides a vector into which an expression cassette has been inserted. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a polynucleotide of the invention into a vector that itself contains regulatory sequences. Thus, an expression cassette is formed upon insertion of the polynucleotide into the vector. Vectors containing regulatory sequences are available commercially and methods for their use are known in the art (Clonetech, Promega, Stratagene).

In the case of a polypeptide or polynucleotide that is naturally occurring, it is preferred that such polypeptide or polynucleotide be isolated and, optionally, purified. An “isolated” polypeptide or polynucleotide is one that is separate and discrete from its natural environment. A “purified” polypeptide or polynucleotide is one that is at least 60% free, preferably 75% free, and most preferably 90% free from other components with which they are naturally associated. Polypeptides and nucleotides that are produced outside the organism in which they naturally occur, e.g., through chemical or recombinant means, are considered to be isolated and purified by definition, since they were never present in a natural environment.

The invention further provides methods for making feline proinsulin, insulin and constituent peptides thereof, analogs, and biologically active subunits; as well as methods for making the polynucleotides that encode them. The methods include biological, enzymatic, and chemical methods, as well as combinations thereof, and are well-known in the art. For example, a feline proinsulin, insulin or constituent peptides can be expressed in a host cell from using standard recombinant DNA technologies; it can be enzymatically synthesized in vitro using a cell-free RNA based system; or it can be synthesized using chemical technologies such as solid phase peptide synthesis, as is well-known in the art. When recombinant DNA technologies are used, the host cell can be, for example, a bacterial cell, an insect cell, a yeast cell, or a mammalian cell. Any cell useful for synthesizing human recombinant insulin is useful for synthesizing feline insulin.

Antibodies and Peptide Aptamers

The invention provides antibodies and peptide aptamers that bind to feline proinsulin, feline insulin, feline insulin A-chain, feline insulin B-chain, feline insulin C-chain, subunits of feline insulin, and analogs thereof. Antibodies, both monoclonal and polyclonal, and peptide aptamers of the invention are particularly useful in diagnostic applications.

Accordingly, feline proinsulin, insulin and constituent peptides as described herein and any portion thereof can be used as antigens to produce antibodies, including vertebrate antibodies, hybrid antibodies, chimeric antibodies, humanized antibodies, altered antibodies, univalent antibodies, monoclonal and polyclonal antibodies, Fab proteins and single domain antibodies. If the polypeptides are not sufficiently immunogenic, they can be modified by covalently linking them to an immunogenic carrier, such as keyhole limpet hemocyanin (KLH), bovine serum albumin, ovalbumin, mouse serum albumin, rabbit serum albumin, and the like.

If polyclonal antibodies are desired, a selected animal (e.g., mouse, rabbit, goat, horse or bird, such as chicken) is immunized with the desired antigen. Serum from the immunized animal is collected and treated according to known procedures. If serum containing polyclonal antibodies to a feline proinsulin, insulin or constituent peptide contains antibodies to other antigens, the polyclonal antibodies can be purified by immunoaffinity chromatography. Techniques for producing and processing polyclonal antisera are known in the art (see for example, Mayer and Walker eds. Immunochemical Methods in Cell and Molecular Biology (Academic Press, London) (1987), Coligan, et al., Unit 9, Current Protocols in Immunology, Wiley Interscience (1991), Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, section 2.4.1 (1992)).

Monoclonal antibodies directed against the polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus (See Monoclonal Antibody Production. Committee on Methods of Producing Monoclonal Antibodies, Institute for Laboratory Animal Research, National Research Council; The National Academies Press; (1999), Kohler & Milstein, Nature, 256:495 (1975); Coligan et al., sections 2.5.1-2.6.7; and Harlow et al., Antibodies: A Laboratory Manual, page 726 (Cold Spring Harbor Pub. 1988)). Panels of monoclonal antibodies produced against the polypeptides of the invention can be screened for various properties, for example epitope affinity.

As an example of one procedure for creating monoclonal antibodies, antigen is emulsified in Complete Freund's Adjuvant and the emulsion used to immunize Balb/c mice (about 50-100 μg antigen per mouse given intraperitoneally). Mice are boosted with an emulsion of antigen-Incomplete Freund's Adjuvant twice at about 10 day intervals (about 50-100 μg antigen each, given intraperitoneally). About ten days after the second booster, an antigen-capture ELISA may be run to determine the response of the mice to the antigen. The ELISA is performed by using the antigen to coat wells of microtiter plates. After overnight incubation, coated plates are washed thoroughly, and nonspecific binding sites are blocked. After incubation, plates are thoroughly washed. The primary antibody, i.e. antibody contained in the sera from the immunized mice, is diluted and added to the microtiter plate wells. Following additional washes, a goat anti-mouse IgG- and IgM- alkaline phosphatase conjugate is added to the wells. After incubation and thorough washing, the substrate for the phosphatase, p-nitrophenyl phosphate, is added to the wells. Plates are incubated in the dark for about 10-45 minutes. Subsequently, changes in absorbance of the plate's contents are read at 405 nm with a microplate spectrophotometer as an indication of mouse response to antigen. With the identification of a positive antibody, production of monoclonal antibodies can proceed. If a positive antibody is not identified, more boosters may be used, or techniques to increase the immunogenicity of the polypeptide can be implemented as stated above.

Responding mice are given a final booster consisting of about 5-100 μg, preferably 25-50 μg of antigen, preferably without adjuvant, administered intravenously. Three to five days after final boosting, spleens and sera are harvested from all responding mice, and sera is retained for use in later screening procedures. Spleen cells are harvested by perfusion of the spleen with a syringe. Spleen cells are collected, washed, counted and the viability determined via a viability assay. Spleen and SP2/0 myeloma cells (ATCC, Rockville, Md.) are screened for HAT sensitivity and absence of bacterial contamination. The screening involves exposing the cells to a hypoxanthine, aminopterin, and thymidine selection (HAT) medium in which hybridomas survive but not lymphocytes or myeloma cells). The cells are combined, the suspension pelleted by centrifugation, and the cells fused using polyethylene glycol solution. The “fused” cells are resuspended in HT medium (RPMI supplemented with 20 % fetal bovine serum (FB S), 100 units of penicillin per ml, 0.1 mg of streptomycin per ml, 100 μM hypoxanthine, 16 μM thymidine, 50 μM 2-mercaptoethanol and 30 % myeloma-conditioned medium) and distributed into the wells of microtiter plates. Following overnight incubation at 37° C. in 5% CO₂, HAT selection medium (HT plus 0.4 μM aminopterin) is added to each well and the cells fed according to accepted procedures known in the art. In approximately 10 days, medium from wells containing visible cell growth are screened for specific antibody production by ELISA. Only wells containing hybridomas making antibody with specificity to the antigen are retained. The ELISA is performed as described above, except that the primary antibody added is contained in the hybridoma supernatants. Appropriate controls are included in each step.

This process generates several hybridomas producing monoclonal antibodies to the feline proinsulin, insulin or constituent peptide antigen. Hybridoma cells from wells testing positive for the desired antibodies are cloned by limiting dilution and re-screened for antibody production using ELISA. Cells from positive wells are subcloned to ensure their monoclonal nature. The most reactive lines are then expanded in cell culture and samples are frozen in 90% FBS-10% dimethylsulfoxide. Monoclonal antibodies can be characterized using a commercial isotyping kit (BioRad Isotyping Panel, Oakland, Calif.) and partially purified with ammonium sulfate precipitation followed by dialysis. Further purification can be performed using protein-A affinity chromatography.

Antibodies can also be prepared through use of phage display techniques. In one example, an organism is immunized with an antigen, such as a polypeptide or coupled polypeptide of the invention. Lymphocytes are isolated from the spleen of the immunized organism. Total RNA is isolated from the splenocytes and mRNA contained within the total RNA is reverse transcribed into complementary deoxyribonucleic acid (cDNA). The cDNA encoding the variable regions of the light and heavy chains of the immunoglobulin is amplified by polymerase chain reaction (PCR). To generate a single chain fragment variable (scFV) antibody, the light and heavy chain amplification products may be linked by splice overlap extension PCR to generate a complete sequence and ligated into a suitable vector. E. coli are then transformed with the vector encoding the scFV, and are infected with helper phage, to produce phage particles that display the antibody on their surface. Alternatively, to generate a complete antigen binding fragment (Fab), the heavy chain amplification product can be fused with a nucleic acid sequence encoding a phage coat protein, and the light chain amplification product can be cloned into a suitable vector. E. coli expressing the heavy chain fused to a phage coat protein are transformed with the vector encoding the light chain amplification product. The disulphide linkage between the light and heavy chains are established in the periplasm of E. coli. The result of this procedure is to produce an antibody library with up to 10⁹ clones. The size of the library can be increased to 10¹⁸ phages by later addition of the immune responses of additional immunized organisms that may be from the same or different hosts.

Antibodies that recognize a specific antigen can be selected through panning. Briefly, an entire antibody library can be exposed to an immobilized antigen against which antibodies are desired. Phage that do not express an antibody that binds to the antigen are washed away. Phage that express the desired antibodies are immobilized on the antigen. These phage are then eluted and again amplified in E. coli. This process can be repeated to enrich the population of phage that express antibodies that specifically bind to the antigen. After phage are isolated that express an antibody that binds to an antigen, a vector containing the coding sequences for the antibody can be isolated from the phage particles and the coding sequences can be recloned into a suitable vector to produce an antibody in soluble form. Phage display methods to isolate antigens and antibodies are known in the art and have been described (Gram et al., Proc. Natl. Acad. Sci., 89:3576 (1992); Kay et al., Phage display of peptides and proteins: A laboratory manual. San Diego: Academic Press (1996); Kermani et al., Hybrid, 14:323 (1995); Schmitz et al., Placenta, 21 Suppl. A:S106 (2000); Sanna et al., Proc. Natl. Acad. Sci., 92:6439 (1995)).

An antibody of the invention may be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described (Orlandi et al., Proc. Nat'l Acad. Sci. USA, 86:3833 (1989) which is hereby incorporated in its entirety by reference). Techniques for producing humanized monoclonal antibodies are described (Jones et al., Nature, 321:522 (1986); Riechmann et al., Nature, 332:323 (1988); Verhoeyen et al, Science, 239:1534 (1988); Carter et al., Proc. Nat'l Acad. Sci. USA, 89:4285 (1992); Sandhu, Crit. Rev. Biotech., 12:437 (1992); and Singer et al., J. Immunol., 150:2844 (1993)).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described (Green et al., Nature Genet., 7:13 (1994); Lonberg et al., Nature, 368:856 (1994); and Taylor et al., Int. Immunol., 6:579 (1994)).

Antibody fragments of the invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described (U.S. Pat. Nos. 4,036,945; 4,331,647; and 6,342,221, and references contained therein; Porter, Biochem. J., 73:119 (1959); Edelman et al., Methods in Enzymology, Vol. 1, page 422 (Academic Press 1967); and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise, an association of V_{H} and V_{L} chains. This association may be noncovalent (Inbar et al., Proc. Nat'l Acad. Sci. USA, 69:2659 (1972)). Alternatively, the variable chains can be linked by an intermolecular disulfide bond or cross-linked by chemicals such as glutaraldehyde (Sandhu, Crit. Rev. Biotech., 12:437 (1992)). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described (Whitlow et al., Methods: A Companion to Methods in Enzymology, Vol. 2, page 97 (1991); Bird et al., Science, 242:423 (1988), Ladner et al., U.S. Pat. No. 4,946,778; Pack et al., Bio/Technology, 11:1271 (1993); and Sandhu, Crit. Rev. Biotech., 12:437 (1992)).

Another form of an antibody fragment is a peptide that forms a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: A Companion to Methods in Enzymology, Vol. 2, page 106 (1991)).

The invention also provides peptide aptamers that bind to the peptides of the invention. Peptide aptamers are peptides that bind to a peptide or polypeptide of the invention with affinities that are often comparable to those for monoclonal antibody-antigen complexes. In one example, peptide aptamers can be isolated according to mRNA display through use of a DNA library that contains a promoter, a start codon, a nucleic acid sequence coding for random peptides, and a nucleic acid sequence that codes for a histidine tag. This library is transcribed using a suitable polymerase, such as T7 RNA polymerase, after which a puromycin-containing poly A sequence is ligated onto the 3′ end of the newly formed mRNAs. When these mRNAs are translated in vitro, the nascent peptides form covalent bonds to the puromycin of the poly A sequence to form an mRNA-peptide fusion molecule. The mRNA-peptide fusion molecules are then purified through use of Ni—NTA agarose and oligo-dT-cellulose. The mRNA portion of the fusion molecule is then reverse transcribed. The double-stranded DNA/RNA-peptide fusion molecules are then incubated with a peptide of the invention and unbound fusion molecules are washed away. The bound fusion molecules are eluted from the immobilized peptides and are then amplified by PCR. This process may be repeated to select for peptide aptamers having high affinity for the polypeptides of the invention. The sequence of the nucleic acid coding for the peptide aptamers can then be determined and cloned into a suitable vector. Methods for the preparation of peptide aptamers have been described (Wilson et al., Proc. Natl. Acad. Sci., 98:3750 (2001)). Accordingly, the invention provides peptide aptamers that recognize polypeptides of the invention.

Antibodies and peptide aptamers can be screened to determine the identity of the epitope to which they bind. An epitope refers to the site on an antigen, such as a polypeptide of the invention, to which the paratope of an antibody binds. An epitope usually consists of chemically active surface groupings of molecules, such as amino acids or sugar side chains, and can have specific three-dimensional structural characteristics, as well as specific charge characteristics. Methods which can be used to identify an epitope are known in the art (Harlow et al., Antibodies: A Laboratory Manual, page 319 (Cold Spring Harbor Pub. 1988).

Antibodies and peptide aptamers may be screened for their ability to specifically bind to a polypeptide of the invention. For example, antibodies or peptide aptamers that specifically bind to feline C-peptide, but not feline proinsulin, can be selected through use of methods routine in the art. Briefly, a buffer containing the antibodies or peptide aptamers can be applied to a column containing immobilized feline C-peptide. The column can be washed to remove antibodies or peptide aptamers that do not bind to the feline C-peptide. The antibodies or peptide aptamers can then be eluted from the column through use of buffer having a high salt concentration. The buffer containing the eluted antibodies or peptide aptamers is then dialyzed to lower the salt concentration. The dialyzed buffer containing the antibodies or peptide aptamers is then applied to a column containing immobilized feline proinsulin. Antibodies or peptide aptamers that bind to the feline proinsulin are retained on the column while antibodies or peptide aptamers which bound the feline C-peptide, but which do not bind the feline proinsulin, will flow through the column. These antibodies or peptide aptamers can be collected and used to specifically detect the feline C-peptide. This procedure can be used with any combination of polypeptides or subunits thereof to select for antibodies and peptide aptamers. Numerous other methods may be used to select antibodies and peptide aptamers that specifically bind to an individual polypeptide or subunit. Such methods are known and are routine to those of skill in the art (see U.S. Pat. No. 6,534,281).

Accordingly, the invention provides antibodies and peptide aptamers that are able to cross-react with feline proinsulin, feline insulin, constituent peptides, and subunits thereof. In addition, the invention provides antibodies and peptide aptamers that are able to specifically bind feline proinsulin, feline insulin, constituent peptides, and subunits thereof, without cross-reacting with other polypeptides.

The antibodies and peptide aptamers of the invention may be coupled to a large variety of detectable markers. Examples of such detectable markers include fluorescent markers, enzymes, radioisotopes, and the like. Methods to couple antibodies and peptide aptamers to detectable markers are known in the art and have been described (see U.S. Pat. No. 6,534,281). Such labeled antibodies and peptide aptamers are useful within automated systems for detection and diagnosis of diabetes within felines.

Pharmaceutical Compositions

The invention provides pharmaceutical compositions that can be used for the administration of polypeptides, peptidomimetics, analogs, antibodies, and peptide aptamers of the invention to a patient in need thereof, such as a feline. In one example, a pharmaceutical composition can contain a polypeptide, peptidomimetic, or analog of the invention, and a pharmaceutically acceptable carrier. In another example, a pharmaceutical composition can contain an antibody or peptide aptamer of the invention, and a pharmaceutically acceptable carrier.

The pharmaceutical compositions of the invention may be prepared in many forms that include tablets, hard or soft gelatin capsules, aqueous solutions, suspensions, and liposomes and other slow-release formulations, such as shaped polymeric gels. An oral dosage form may be formulated such that the polypeptide, coupled polypeptide, antibody, or peptide aptamer is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

A polypeptide, coupled polypeptide, antibody, or peptide aptamer can be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents.

Pharmaceutical compositions suitable for rectal administration can be prepared as unit dose suppositories. Suitable carriers include saline solution and other materials commonly used in the art.

For administration by inhalation, a polypeptide, peptidomimetic, coupled polypeptide, antibody or peptide aptamer can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, a polypeptide, peptidomimetic, coupled polypeptide, antibody, or peptide aptamer may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, a polypeptide, antibody, peptidomimetic, peptide aptamer may be administered via a liquid spray, such as via a plastic bottle atomizer.

A polypeptide, coupled polypeptide, antibody, or peptide aptamer can be formulated for transdermal administration. A polypeptide, coupled polypeptide, antibody, or peptide aptamer can also be formulated as an aqueous solution, suspension or dispersion, an aqueous gel, a water-in-oil emulsion, or an oil-in-water emulsion. A transdermal formulation may also be prepared by encapsulation of a polypeptide, coupled polypeptide, antibody, or peptide aptamer within a polymer, such as those described in U.S. Pat. No. 6,365,146. The dosage form may be applied directly to the skin as a lotion, cream, salve, or through use of a patch. Examples of patches that may be used for transdermal administration are described in U.S. Pat. Nos. 5,560,922 and 5,788,983.

Pharmaceutical compositions of the invention may also contain other ingredients such as flavorings, colorings, anti-microbial agents, and preservatives. In addition, a pharmaceutical composition of the invention can include pharmaceutically active ingredients, such as hormones, anti-necrotic agents, vasodilators, and the like. Insulin is a preferred hormone.

It will be appreciated that the amount of a polypeptide, peptidomimetic, coupled polypeptide, antibody, or peptide aptamer required for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form.

Diagnostic Methods

The invention provides methods to diagnose diabetes. In a preferred embodiment, the diagnostic method of the invention is an immunoassay. Purified feline proinsulin, feline insulin, and any of the constituent peptides of feline proinsulin or insulin are useful as polypeptide standards in an immunoassay. The assay utilizes one or more antibodies induced against an epitope of any of these compounds in a competitive and non-competitive assay such as a radioimmunoassay, immunoenzymometric assay, immunofluorometric assay or enzymoimmunassays assays, or in chemiluminescent methods with horseradish peroxidase or alkaline phosphatase or other chemiluminescent detection agents to analyze, for example, proinsulin, insulin or C-peptide concentrations in plasma, serum or urine. These assays can be used to measure the amount of circulating proinsulin; insulin; constituent peptides; and A-, B-, and C-peptide in plasma, serum or urine.

The polypeptide standards and antibodies can also be used for Western blotting, in the chromatographic analysis of all of the compounds, and in bioassays (e.g. to measure an increase of glycogen of isolated rat diaphragm in glucose medium and glucose uptake by the rat epididymal fat pad) (see, e.g., L. Vu et al., Anal. Biochem.1998, 262: 17-22; A. Christopher et al., Indian J. Med. Res. 1974, 62: 1499-1510; A. Moody, Experientia 1964, 20: 646-648; K. Gundersen et al., Diabetes 1965, 14: 805-810). They can also be used in radioreceptor bioassay which is based on the competition of labeled and unlabeled hormone for binding to a specific tissue receptor (such as rat erythrocyte membrane, fat or liver cells) (K. Gambhir et al., Biochem. Med. Metab. Biol. 1991, 45: 133-153; M. Laburthe et al., Diabetologia 1975, 11: 517-526).

The results from the immunoassay or bioassay can be used to determine the total amount of proinsulin, constituent peptides, and individual chains. In the case of insulin and proinsulin, the ratio of the 2 hormones has been shown to be of value as a predictor for the progression to diabetes. Relative and absolute concentrations of the hormones can be used diagnostically. (L Mykkanen et al., Diabetologia 38: 1176-1182, 1995). Relative and absolute concentrations of both hormones are used diagnostically (N. Wareham et al., Diabetes Care 1999, 22: 262-270; A. Hanley et al., Diabetes 2002; 51: 1263-1270). In Wareham et al., the relative risk to develop diabetes (top quartile) occurred when fasting total proinsulin (proteolytically cleaved and intact) values were >13 pmol/l; fasting intact proinsulin >4.9 pmol/l; fasting proteolytically cleaved proinsulin >8.7 pmol/l and the ratio of proinsulin to insulin >0.34.

The invention also provides a method to assess the predisposition of a feline with type-2 diabetes mellitus to develop neuropathy, retinopathy and nephropathy. The method is based on the reported correlation of low insulin C-peptide concentrations (<0.7 ng/ml) with the development of neuropathy, retinopathy and nephropathy in humans (Inukai et al., Exp. Clin. Endocrinol. Diabetes, 107:40 (1999)). Accordingly, the level of circulating C-peptide in a biological sample obtained from a feline can be determined to indicate whether the feline is predisposed toward developing neuropathy, retinopathy and nephropathy. The C-peptide concentration that is indicates the predisposition of a feline to develop neuropathy, retinopathy and nephropathy can be determined using reported methods (Inukai et al., Exp. Clin. Endocrinol. Diabetes, 107:40 (1999)). Immunological methods as described herein are a preferred method of determining insulin C-peptide concentration. However, additional methods may be used to practice the invention.

Method to Screen for an Antiproliferative Factor

The invention provides a method to screen for an antiproliferative factor. Such a factor is thought to be particularly useful for treating neuroblastoma. The method relates to the finding that the insulin C-peptide in the presence of insulin exerts synergistic effects on cell proliferation, neurite outgrowth, and has an antiapoptotic effect on neuroblastoma cells (Li et al., Diabetes Metab. Res. Rev., 19:375 (2003)).

Generally, the method includes assays that can be used to determine if a candidate factor disallows productive interaction of the insulin C-peptide with the insulin receptor. It has been shown that productive interaction of the C-peptide with the insulin receptor in the presence of insulin causes increased autophosphorylation of the insulin receptor. Accordingly, productive interaction of the insulin C-peptide with the insulin receptor can be determined by measuring autophosphorylation of the insulin receptor in the presence of insulin and the C-peptide. Such methods can be used to confirm the specific interaction of the C-peptide with the insulin receptor.

The antiproliferative factor is thought to bind to the insulin receptor through mimicking the structure of the C-peptide, without stimulating the insulin receptor. Thus, the antiproliferative factor will act as a competitive inhibitor of C-peptide binding by the insulin receptor. Accordingly, the structure of the insulin C-peptide provides guidance to those of skill in the art for development of the antiproliferative factor. Thus, a polynucleotide encoding a peptide having SEQ ID NO. 9 can be randomly mutagenized and expressed within a cell. The resulting peptides can be isolated and purified according to standard procedures, and those described herein. The purified peptides can then be screened for their ability to block interaction of the C-peptide with the insulin receptor, and their inability to stimulate the insulin receptor. Methods for expression and purification methods are well known in the pharmaceutical industry.

The structure of any antiproliferative factors can be determined and then further modified through the replacement of naturally occurring amino acids with amino acid analogs, and the creation of peptidomimetics. The antiproliferative activity of these derivatives can be further determined through use of the methods described herein and known in the art.

In another example, a cellular proliferation assay can be used to identify antiproliferative agents. In this assay, neuroblastoma test cells are incubated with insulin, C-peptide, and a candidate antiproliferative agent. Neuroblastoma control cells are incubated with insulin and C-peptide. Proliferation of the test cells in the presence of the candidate antiproliferative agent can then be compared to proliferation of the control cells to determine if the candidate agent reduced cellular proliferation. The candidate agent can be further screened for the ability to block productive interaction of the C-peptide with an insulin receptor. Numerous neuroblastoma cell lines are commercially available from the American Type Tissue Culture Collection, Manassas, Virginia. Examples of such cell lines have the following ATCC numbers: HB-8437, HB8568, HB-8767, HTB-10, HTB-11, TIB-198. Many additional neuroblastoma cell lines are known in the art and are available.

Additional assays can be used to identify a candidate agent that blocks the action of the insulin C-peptide. These assays include determining if the candidate agent can induce apoptosis, block neurite outgrowth, reduce the stimulation of phosphoinositide 3-kinase, reduce p38 mitogen-activated protein kinase activation, decrease expression and nuclear translocation of nuclear factor kappaB, reduce expression of Bcl2, increase c-jun N-terminal kinase phosphorylation, reduce stimulation of the Na(+),K(+)-ATPase activity, and reduce the stimulation of nitric oxide synthase activity resulting from the action of the C-peptide. The ability of the candidate agent to reduce or eliminate the activity of the C-peptide can then be further confirmed through use of the proliferation assay and insulin autophosphorylation assays as described herein.

Therapeutic Applications

Diabetes mellitus in the cat and in other species leads to an impairment of insulin secretion. Glucose production from precursors (fat, protein) is increased, while glucose uptake into most cells, except red blood cells, platelets, neural, ocular cells and few others is decreased. The subsequent increase in blood glucose leads to an osmotic diuresis and polyuria when it exceeds the renal threshold and the increased water loss into the urine results in a compensatory polydipsia. Diabetic cats may have enlarged livers due to fatty infiltration and about one third of all diabetic cats presents with icterus. The haircoat may be unkempt. Other clinical signs may include anorexia, dehydration, depression, and vomiting, especially in the sick ketoacidotic animal, and neuropathy which is manifested by a plantigrade stance. Additional examples of diabetes associated disorders include nerve dysfunction resulting from diabetic neuropathy, hippocampal apoptosis, decreased blood flow, decreased muscle glucose utilization, glomerular hypertrophy, renal hypertrophy, glomerular hyperfiltration, and urinary albumin excretion.

Treatment of diabetes must be adjusted to the clinical presentation of the cat. Diabetic cats can be treated with feline insulin. The goal of treatment of any diabetic is to maintain blood glucose concentrations in a mild hyperglycemic state, i.e. the blood glucose should stay between 150 and 200-250 mg/dl for most of the day and should not drop into the low normal range. This will eliminate the increased water intake and urination and other clinical signs due to insulin deficiency and/or hyperglycemia and prevent the dangers of hypoglycemia. Because every animal reacts differently to a given dose of insulin, the insulin treatment needs to be adjusted to the individual's needs with regard to dose of insulin and frequency of administration.

An example of an initial treatment regimen in diabetic cats is to give insulin (0.25-0.5 units/kg body weight, where the units are based on commercially available insulin preparations), subcutaneously in the morning and follow the change in blood glucose concentrations. This insulin dose is a low dose which may have to be adjusted after a few days should the glucose control be unsatisfactory (i.e. a glucose concentration >250 mg/dl for most of the day). The insulin dose is preferably increased (if the blood glucose stays too high) or decreased (if the blood glucose drops too low) by 10-25%. Glucose concentrations can be monitored with individual blood or urine glucose measurements. Long-term glucose control can also be assessed with fructosamine and glycosylated hemoglobin measurements.

The invention provides a method to reduce or ameliorate nerve dysfunction in a feline resulting from diabetic neuropathy. The method is based on the finding that insulin C-peptide stimulates the activities of Na(+),K(+)-ATPase and nitric oxide synthase. Both of these enzyme systems are known to be important for nerve function (Li et al., Diabetes. Metab. Res. Rev., 19:375 (2003), Cotter et al., Diabetes, 52:1812 (2003), Ekberg et al., Diabetes, 52:536 (2003), Forst et al., Exp. Clin. Endocrinol. Diabetes, 106:270 (1998)). Accordingly, the method involves administering an effective amount of a feline insulin C- peptide, or an analog or peptidomimetic thereof, to the feline in need of such treatment. The feline insulin C-peptide, subunit, analog, or peptidomimetic may be administered alone, or as a pharmaceutical composition.

The invention provides a method to reduce or ameliorate onset of hippocampal apoptosis in type 1 diabetic felines. The method is based on the finding that insulin C-peptide has an antiapoptotic effect on neural cells when administered in the presence of insulin (Li et al., Diabetes. Metab. Res. Rev., 19:375 (2003)). Accordingly, the method involves administering an effective amount of insulin and a feline insulin C-peptide, or an analog or peptidomimetic thereof, to the feline in need of such treatment. The feline insulin C-peptide, analog, or peptidomimetic may be administered alone, or as a pharmaceutical composition.

Administration of the insulin C-peptide has been demonstrated to increase blood flow in a mammal to skeletal muscle, myocardium, skin, and nerve tissues (Wahren and Jornvall, diabetes Metab. Res. Rev., 19:345 (2003), Cotter et al., Diabetes, 52:1812 (2003), Forst et al., Exp. Clin. Endocrinol. Diabetes, 106:270 (1998)). Administration of insulin C-protein also produces increased muscle glucose utilization (Forst et al., Exp. Clin. Endocrinol. Diabetes, 106:270 (1998); decreases glomerular and renal hypertrophy (Wahren et al., Curr. Diab. Rep., 1:261 (2001), and reduces glomerular hyperfiltration and urinary albumin excretion (Wahren et al., Curr. Diab. Rep., 1:261 (2001)).

Accordingly, an effective amount of feline C-peptide, or an analog or peptidomimetic thereof, may be administered therapeutically to a feline to increase blood flow, increase muscle glucose utilization, decrease glomerular and renal hypertrophy, reduce glomerular hyperfiltration, and reduce urinary albumin excretion.

The amount of feline C-peptide, peptidomimetic, or analog that is effective to achieve a desired result will depend upon the route and pharmaceutical formulation used for administration. While the feline C-peptide is thought to be active at nanomolar concentration, the dosage of the feline insulin C-peptide, analog, or peptidomimetic can be determined at the time of administration by a person of skill in the veterinary arts.

Kits

The invention provides kits that contain reagents used for diagnosing diabetes in a feline. Such kits can contain packaging material, and an antibody, peptide aptamer, or both an antibody and peptide aptamer that bind to feline C-peptide. Such kits may also be used by medical personal for the formulation of pharmaceutical compositions that contain an antibody or peptide aptamer of the invention.

The packaging material will provide a protected environment for the antibody or peptide aptamer. For example, the packaging material may keep the antibody or peptide aptamer from being contaminated. In addition, the packaging material may keep an antibody or peptide aptamer in solution from becoming dry.

Examples of suitable materials that can be used for packaging materials include glass, plastic, metal, and the like. Such materials may be silanized to avoid adhesion of an antibody or peptide aptamer to the packaging material.

In one example, the invention provides a kit that includes a first antibody that specifically binds to the feline C-peptide, a second antibody that specifically binds to feline insulin, and packaging material. The kit may optionally include additional components such as buffers, reaction vessels, secondary antibodies, and syringes. In one example, a kit can include a first antibody that specifically binds to the feline C-peptide, a second antibody that specifically binds to feline insulin, a syringe, a tray to which the first or second antibody can be immobilized, wash buffer, and packaging material.

In another example, the invention provides a kit that includes a first antibody that specifically binds to the C-peptide portion of feline proinsulin, a second antibody that specifically binds to feline insulin, and packaging material. The kit may optionally include additional components such as buffers, reaction vessels, secondary antibodies, and syringes. For example, a kit can include the first antibody that specifically binds to the C-peptide portion of feline proinsulin, a second antibody that specifically binds to feline insulin, a syringe, a tray to which the first or second antibody can be immobilized, wash buffer, and packaging material.

The present invention is illustrated by the following examples. It is to be understood that the particular examples, materials, amounts, and procedures are to be interpreted broadly in accordance with the scope and spirit of the invention as set forth herein.

### EXAMPLES

### Example I

Cloning of Feline Proinsulin (Proinsulin-met) into pET21b Vector

Primers were designed based on alignment of known proinsulin cDNA sequences from other species. Most of the primers contained degeneracies to accommodate regions of species variability in the alignment. The cDNA was transcribed from feline pancreatic messenger RNA using a non-specific T-tailed primer (EPB 1 8T; 5′-GCG AAT TCT GCA GGA TCC AAA CTT TTT TTT TTT TTT TTT T-3′; SEQ ID NO:17), then amplified by routine PCR with a specific sense primer (INS1; 5′-CCT GCC CCG ACC CGA GCC TTC GTC AAC-3′; SEQ ID NO:18) and the non-specific primer EPB 18T. Both primers were synthesized by Molecular Genetics Instrumentation Facility University of Georgia, Athens, Ga.

The resulting PCR product was sequenced from each end using INS1 and EPB 18T as primers, and the sequence of the entire coding region for the mature form of feline proinsulin, which encompasses the coding sequence for the A, B, and C chain, was obtained. This proinsulin sequence was functionally organized similar to other mammalian proinsulins as follows, from the 5′ to the 3′ direction: 5′ NTR-signal peptide coding region with intron, followed by the B-chain coding region, followed by the C-chain coding region with intron, followed by the A chain coding region, followed by a stop codon, followed by 3′NTR (NTR; nontranslated region). As with all proteins produced with pET vectors, the proinsulin includes a methionine at the 5′ end of the expressed protein. See Example III for production of proinsulin without the 5′ methionine.

The coding regions for the three contiguous feline proinsulin constituent chains (including the dipeptide connector sequences) are shown below.

| | |
|---|---|
| B chain: | (SEQ ID NO: 19) |

5′ TTC GTC AAC CAG CAC CTG TGC GGC TCC CAC CTG GTG
GAG GCG CTG TAC CTG GTG TGC GGG GAG CGC GGC TTC TTC
TAC ACG CCC AAG GCC 3′

C-chain (including the codons that encode the dipeptide linkers, which are subsequently cleaved off the C-chain during polypeptide processing):

| | |
|---|---|
| 5′ CGC CGG GAG | (SEQ ID NO: 20) |

GCG GAG GAC CTC CAG GGG AAG GAC GCG GAG CTG GGG GAG
GCG CCT GGC GCC GGC GGC CTG CAG CCC TCG GCC CTG GAG
GCG CCC CTG CAG AAG CGG 3′

| | |
|---|---|
| A chain: | (SEQ ID NO: 21) |

5′ GGC ATC GTG GAG CAA TGC TGT GCC AGC GTC TGC TCG
CTG TAC CAG CTG GAG CAT TAC TGC AAC 3′

The coding region for proinsulin includes all three constituent peptide coding regions:

(SEQ ID NO: 22)

5′ TTC GTC AAC CAG CAC CTG TGC GGC TCC CAC CTG GTG
GAG GCG CTG TAC CTG GTG TGC GGG GAG CGC GGC TTC TTC
TAC ACG CCC AAG GCC CGC CGG GAG GCG GAG GAC CTC CAG
GGG AAG GAC GCG GAG CTG GGG GAG GCG CCT GGC GCC GGC
GGC CTG CAG CCC TCG GCC CTG GAG GCG CCC CTG CAG AAG
CGG GGC ATC GTG GAG CAA TGC TGT GCC AGC GTC TGC TCG
CTG TAC CAG CTG GAG CAT TAC TGC AAC 3′.

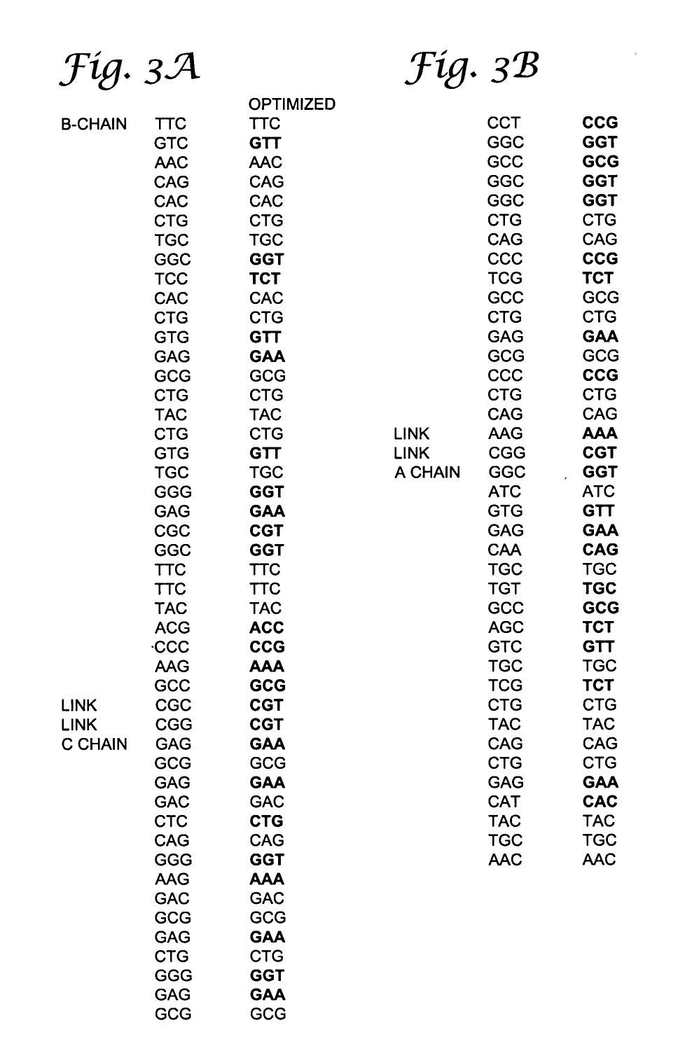

Isolation of this PCR product made it possible to determine the nucleotide sequence of feline proinsulin (FIG. 3). A synthetic feline proinsulin DNA sequence was then prepared (Molecular Genetics Instrumentation Facility University of Georgia, Athens, GA) to reflect optimization of the codons for the subsequent expression in E. coli (SEQ ID NO:33) (Henaut et al., in Escherichia coli and Salmonella, Vol. 2, Ch. 114:2047-2066, 1996, Niedhardt, FC ed., ASM press, Washington, D.C.) (FIG. 3). Based on this optimized sequence new primers were designed containing specific restriction sites for NdeI at the 5′ end and BamHI at the 3′ end which allowed for directional cloning into pCR 2.1—Topo (Invitrogen, Carslbad, Calif.). The sequence of the newly designed 5′ primer was: 5′-CTC CAT ATG TTC GTT AAC CAG CAC CTG-3′ (SEQ ID NO:23); the sequence of the newly designed 3′ primer was: 5′-GCG GGA TCC CTA GTT GCA GTA GTG TTC CAG-3′ (SEQ ID NO:24). Both primers were synthesized by Genemed Synthesis, Inc (San Francisco, Calif.).

After routine PCR of the entire synthetic feline proinsulin coding sequence, the amplified DNA was analyzed by agarose (1.8%) gel electrophoresis to check for purity and proper size of the amplified product. The DNA was extracted using Quantum Prep Freeze n'Squeeze DNA Gel Extraction Spin Columns (Bio-Rad, Hercules, Calif.) and cloned into the plasmid pCR 2.1—TOPO (Invitrogen, Carlsbad, Calif.). DH5α cells (Invitrogen, Carlsbad, Calif.) were used to transform the intact plasmid. The vector carries a short segment of E. coli DNA that contains the regulatory sequences and the coding information of the β-galactosidase gene. Although neither the host-encoded nor the plasmid-encoded fragments are themselves active they can associate to form an enzymatically active protein. The Lac+ bacteria that result from beta-complementation are easily recognized because they form blue colonies in the presence of the chromogenic substrate 5-bromo-4-chloro-3-indolyl-β-D-galactoside (X-gal). However insertion of a fragment of foreign DNA into the polycloning site of the plasmid almost invariably results in production of an amino-terminal fragment that is not capable of β-complementation. Bacteria carrying recombinant plasmids therefore form white colonies. Successful cloning was therefore evident by the growth of white colonies Plasmid DNA from positive clones was isolated using the high pure plasmid isolation kit (Roche, Indianapolis, Ind.).

The plasmid and an expression vector, pET 21b (Novagen, Madison Wis.) were cut with the appropriate endonucleases, NdeI and BamHI. The cut plasmid insert and pET 21b vector were isolated using agarose (1.8%) gel electrophoresis. The DNA was extracted using Quantum Prep Freeze n'Squeeze DNA Gel Extraction Spin Columns (Bio-Rad, Hercules, Calif.). The cut plasmid insert was then cloned into the pET21b vector using the rapid DNA Ligation Kit (Roche, Indianapolis, Ind.). The ligations were performed at room temperature for 15 minutes. This pET vector was chosen because it contains a T7 -Tag which can be used in the purification process.

The new plasmid construct was transformed into BL21 (Gold) (DE3) competent cells according to the protocol (Novagen, Madison, Wis.). Transformed bacteria were grown at 37° C. on LB/ampicillin plates and individual colonies were picked and grown overnight at 37° C. in 5 ml LB broth containing 100 μg/ml ampicillin. An aliquot of the plasmid DNA was digested with the endonucleases NdeI and BamHI and loaded onto a 1.8% agarose gel to verify the presence of the proinsulin cDNA insert. The proinsulin sequence was verified by automated DNA sequencing (Molecular Genetics Instrumentation Facility University of Georgia, Athens, Ga.).

Expression Offeline Proinsulin

Two ml of the overnight culture were added to inoculate 500 ml of LB/ampicillin broth. The culture was grown in a shaking incubator at 37° C. until OD₆₀₀ 0.6-0.8. Protein expression was induced by adding isopropyl β-D-thiogalactopyranoside (IPTG) to a final concentration of 0.5 mM. The culture was grown for an additional for 2.5 hours. Pre-and post IPTG protein expression was analyzed using sodium dodecyl sulfate-polyacrylamide gel electrophoresis (SDS-PAGE).

Purification, Reduction and Refolding Offeline Proinsulin Expressed in pET 21b

Proinsulin expressed in pET 21b was purified as outlined in the pET system manual (Novagen, Madison, Wis.) using the BugBuster/benzonase inclusion body purification protocol. The resulting pellet was resuspended in 5 ml/g (original weight of bacteria) 70% formic acid and incubated for 10-15 minutes. The sample was centrifuged at 10,000 g for 10 minutes at 20° C. The supernatant was applied to a Bio-Gel P-2 equilibrated with 200 mM Tris/HCl, 8M urea/5mM EDTA pH 8.7. The elution of protein was monitored at 280nm at a flow rate of 1.0 ml/minute and all peaks were collected. Protein concentration of the peaks was determined using the Bradford protein assay. The protein peak is the first peak in the elution profile.

The protein was further processed according to the method described by Mackin and Choquette (R. Mackin et al., Protein Expr. Purific., 27:210-219 (2003)). The purification involves reduction of the protein with dithiothreitol (60 mM final concentration). Argon was bubbled into the solution, the tube was filled with argon and the cap tightened immediately. The sample was incubated at 50° C. for 30 minutes, then cooled to room temperature and applied to the same Bio-Gel P-2 column which was equilibrated with 50 mM glycine/NaOH, pH 10.5, 1 mM EDTA, flow rate of 2 ml/minute. The protein peak was collected.

The amount of proinsulin was determined using reverse phase high performance liquid chromatography (RP-HPLC) and the peptide was diluted to a final protein concentration of 100 μg/ml using fresh 50 mM glycine/NaOH, pH 10.5, 1 mM EDTA buffer. Both reduced and oxidized glutathione were added to a final concentration of 1 mM each. The sample was briefly bubbled with argon, the tube filled with argon and immediately sealed. The sealed sample was incubated at 4° C. overnight.

Folded proinsulin was purified using a reverse phase column (Jupiter C4 4.6×250 mm; Phenomenex, Torrance, Calif.). The column was equilibrated with 75% H₂O/25% acetonitrile. The sample was prepared for injection to contain a final solution of 0.1% TFA, 4% acetonitrile and 100 mM HCL. The filtered sample was injected and eluted using a linear gradient of 0.1 % trifluoroacetic acid (TFA) (A) and 0.1% TFA in 20% H₂O/80% acetonitrile (B) increasing B from 25% to 100% in 32 minutes. Human recombinant proinsulin (Eli Lilly, Indianapolis, Ind.) was used as a standard. The purity of feline proinsulin was determined by mass-spectroscopy (Proteomics Facility, University of Georgia, Athens, Ga.).

Feline proinsulin and human proinsulin have 72 amino acids out of 86 in common, for an overall percentage identity of about 84% (see FIG. 1). It has the same number of amino acids as human proinsulin, whereas pork and beef proinsulin both have deletions in the C-chain compared to human and feline proinsulin. Feline proinsulin constituent peptide sequences, in comparison to the human sequences, are as follows (N- terminal to C-terminal sequences):

B chain (30 amino acids):

| | |
|---|---|
| Feline(1-30): | (SEQ ID NO: 5, FIG. 1) |
| FVNQHLCGSHLVEALYLVCGERGFFYTPK | |
| A | |
| Human(1-30): | (SEQ ID NO: 6, FIG. 1) |
| FVNQHLCGSHLVEALYLVCGERGFFYTPK | |
| T | |

Feline and human B chain amino acid sequences differ only at one position: position 31 (30/31=96.7% identity). However, the feline B chain is identical to the B chains in pork and beef insulin (see FIG. 1).

C chain (31 amino acids; coding region encodes 35 amino acids including the initial Arg- Arg linker dipeptide and the terminal Lys-Arg dipeptide which are cleaved off during processing):

Feline(31-65):
(RR)EAEDLQGKDAELGEAPGAGGLQPSAL
EAPLQ(KR)
(C-peptide includes amino acids 33-63,
SEQ ID NO: 9, FIG. 1)
Human(31-65):
(RR)EAEDLQVGQVELGGGPGAGSLQPLAL
EGSLQ(KR)
(C-peptide includes amino acids 33-63,
SEQ ID NO: 10, FIG. 1)

Feline and human C chain amino acid sequences differ at ten positions (21/31=67.7% identity).

A-chain (21 amino acids):

| | |
|---|---|
| Feline(66-86): | |
| GIVEQCCASVCSLYQLEHYCN | (SEQ ID NO: 13, FIG. 1) |
| Human(66-86): | |
| GIVEQCCTSICSLYQLENYCN | (SEQ ID NO: 14, FIG. 1) |

Feline and human A chain amino acid sequences differ at 3 positions (18/21=85.7% homology). However, the feline A chain differs only at one position from the A chain in beef and at 3 positions from the A chain in pork insulin (FIG. 1), yielding 95.2% amino acid sequence identity with the beef A chain.

Proinsulin was converted to insulin and C-peptide using the method described by Kemmler et al. (J. Biol. Chem. 246: 6780-6791, 1971). Briefly, proinsulin was incubated with trypsin (25 μg/ml; Sigma, St. Louis, Mo.) and carboxypeptidase B (12.5 μg/ml; Worthington, Lakewood, N.J.) at 37° C. for 10 minutes in 0.1 M Tris-HCl buffer, pH 7.6. Insulin and C-peptide were purified using RP-HPLC as described above.

### Example II

Cloning of Feline Proinsulin into pET28 Vector

In this example, feline proinsulin was cloned into pET28 vector which is designed to drive expression in E. coli and add a 6-histidine tag to the product to facilitate purification. Primers were designed containing a factor XA cutting site and specific restriction sites for NdeI at the 5′ end and EcoRI at the 3′ end which would allow directional cloning cloning into pCR 2.1—TOPO (Invitrogen). The sequence of the 5′ primer was: 5′-GGA TCC CAT ATG ATC GAA GGT CGT TTC GTC AAC CAG CAC CTG TGC-3′ (SEQ ID NO:25). The sequence of the 3′ primer was: 5′-CGG AAT TCC TAG TTG CAG TAG TGT TCC AGC TG-3′ (SEQ ID NO:26). The primers were synthesized by the Molecular Genetics Instrumentation Facility University of Georgia, Athens, Ga.

After routine PCR of the entire feline proinsulin coding sequence, the amplified DNA was analyzed by agarose (1.8%) gel electrophoresis to check for purity and proper size of the amplified product. The DNA was extracted using Quantum Prep Freeze n' Squeeze DNA Gel Extraction Spin Columns (Bio-Rad, Hercules, Calif.) and cloned into pCR 2.1—Topo. Successful cloning was evident by the growth of white colonies, as described in Example I. Plasmid DNA from positive clones was isolated using the high pure plasmid isolation kit (Roche, Indianapolis, Ind.).

The plasmid and pET 28 expression vector (Novagen, Madison, Wis.) were cut with the appropriate endonucleases, NdeI and EcoRI. The vector was dephosphorylated by treatment with calf intestinal phosphatase, and the cut PCR fragment was then cloned into the pET28 vector using the rapid DNA Ligation Kit (Boehringer Mannheim). Optionally, the vector was gel purified without being dephosphorylated and used within the ligation reaction. The ligations were for performed at room temperature for 15 minutes. The new plasmid construct was transformed into BL21Gold (DE3) competent cells according to the protocol (Novagen, Madison, Wis.). Transformed bacteria were grown at 37° C. on LB/Kanamycin plates and individual colonies were picked and grown overnight at 37° C. in 5 ml LB broth containing 50 μ/ml kanamycin.

An aliquot of the plasmid DNA was digested with the endonucleases NdeI and Eco RI and loaded onto a 1.8% agarose gel to verify the presence of the proinsulin cDNA insert. The proinsulin sequence was verified by automated DNA sequencing (Molecular Genetics Instrumentation Facility University of Georgia, Athens, Ga.).

Expression Offeline Proinsulin

Two ml of the overnight culture were added to inoculate 500 ml of LB/kanamycin broth. The culture was grown in a shaking incubator at 37° C until OD₆₀₀ 0.6-0.8. Protein expression was induced by adding isopropyl-β-D-thiogalactopyranoside (IPTG) to a final concentration of 0.5 mM. The culture was grown for an additional hour. Pre-and post IPTG protein expression was analyzed using sodium dodecyl sulfate-polyacrylamide gel electrophoresis (SDS-PAGE).

Purification, Reduction and Refolding Offeline Proinsulin Expressed in pET28

Proinsulin expressed in pET 28 was purified as outlined in the pET system manual (Novagen, Madison, Wis.). Cells were lysed using either the BugBuster/bensonase inclusion body protocol (as described above in Example I for proinsulin. expressed in pET21) or a french press. The protein preparation was applied to a chelating sepharose fast flow column (Amersham, Piscataway, N.J.) or a HiTrap chelating HP columns (Amersham, Piscataway, N.J.). Polypeptides were eluted with imidazole. Fractions were assayed for protein content and further analyzed with SDS page electrophoresis.

Because factor Xa activity is inhibited by imidazole, the protein was dialyzed overnight in Tris-buffer before incubation with factor Xa (37° C. for 30 minutes). Cleavage was monitored with SDS polyacrylamide gel electrophoresis. Factor Xa was removed during the protein purification step using RP-HPLC. The resulting proinsulin was then further processed (folding procedure) as outlined in Example I, or it can be converted to insulin and C-peptide as outlined in Example I (method described by Kemmler et al. J. Biol. Chem. 246: 6780-6791, 1971).

### Example III

Cloning of Feline Proinsulin Containing FactorXa Cleavage Site Into pET21b Vector

In this example, feline proinsulin was cloned into pET21b vector. Primers were designed containing a factor Xa cutting and specific restriction sites for NdeI at the 5′ end and BamHI at the 3′ end which would allow directional cloning into pCR 2.1—Topo (Invitrogen, Carlsbad, Calif.). The factor Xa cutting site allows the elimination of the methionine at the 5′ end of the expressed protein. The sequence of the 5′ primer was: 5′-GGA TCC CAT ATG ATC GAA GGT CGT TTC GTT AAC CAG CAC CTG TGC-3′ (SEQ ID NO:25), synthesized by Molecular Genetics Instrumentation Facility University of Georgia, Athens, Ga.). The sequence of the 3′ primer was: 5′-GCG GGA TCC CTA GTT GCA GTA GTG TTC CAG-3′ (SEQ ID NO:24), synthesized by Genemed Synthesis, Inc, San Francisco, Calif. Cloning and expression were done as described in Example I for proinsulin-met. The terminal methionine was removed with factor Xa (Novagen, Madison, Wis.). Briefly, the peptide was incubated with Factor Xa at 37° C. for 30 minutes using 0.5 U per 10 μg of protein.

Cleavage can be monitored with SDS polyacrylamide gel electrophoresis. After cleavage, the peptide is ready for purification.

### Example IV

Cloning of the Feline A-, B-, and C-Chains into pET28 Vector

The constituent chains of feline proinsulin were each individually cloned into pET28 vector. Primers were designed based on the feline proinsulin sequence containing a factor XA cutting site and specific restriction sites for NdeI at the 5′ end and EcoRI at the 3′ end which would allow directional cloning cloning into pCR 2.1—TOPO (Invitrogen, Carlsbad, Calif.). The sequences of primers were:

| | |
|---|---|
| A-chain 5′ primer: | (SEQ ID NO: 27) |

5′-GGA TCC CAT ATG ATC GAA GGT CGT GGT ATC
GTT GAA CAG TGC TGC GC-3′

| | |
|---|---|
| A-chain 3′ primer: | (SEQ ID NO: 26) |

5′-CGG AAT TCC TAG TTG CAG TAG TGT TCC AGC TG-3′

| | |
|---|---|
| B-chain 5′ primer: | (SEQ ID NO: 25) |

5′-GGA TCC CAT ATG ATC GAA GGT CGT TTC GTT
AAC CAG CAC CTG TGC-3′

| | |
|---|---|
| B-chain 3′ primer: | (SEQ ID NO: 28) |

5′-CGG AAT TCT ACG CTT TCG GGG TGT AGA AGA
AAC C-3′

(SEQ ID NO: 29)

C-chain 5′ primer (with dipeptide linkage):
5′-GGA TCC CAT ATG ATC GAA
GGT CGT CGT CGT GAA GCG GAA GAC CTG-3′

(SEQ ID NO: 30)

C-chain 3′ primer (with dipeptide linkage):
5′-CGG AAT TCC TAA CGT TTC
TGC AGC GGC GCT TC-3′

(SEQ ID NO: 31)

C-chain 5′ primer (without dipeptide linkage):
5′-GGA TCC CAT ATG ATC
GAA GGT CGT GAA GCG GAA GAC CTG CAG GGT-3′

(SEQ ID NO: 32)

C-chain 3′ primer (without dipeptide linkage):
5′-CGG AAT TCC TAC TGC
AGC GGC GCT TCC AGC GCA GAC GG-3′

Primers were synthesized at the Molecular Genetics Instrumentation Facility University of Georgia, Athens, Ga. After routine PCR, the amplified DNA of each of the chains was analyzed by agarose (1.8%) gel electrophoresis to check for purity and proper size of the amplified product. The DNA was extracted using Quantum Prep Freeze n' Squeeze DNA Gel Extraction Spin Columns (Bio-Rad, Hercules, Calif.) and cloned into pCR 2.1—TOPO. Successful cloning was evident by the growth of white colonies as described in Example I. Plasmid DNA from positive clones (for each chain) was isolated using the high pure plasmid isolation kit (Roche, Indianapolis, Ind.).

The respective plasmids and pET28 expression vector (Novagen, Madison, Wis.) were cut with the appropriate endonucleases, NdeI and EcoRI. The vector was dephosphorylated by treatment with calf intestinal phosphatase, and the cut PCR fragments were then cloned into the pET28 vector using the rapid DNA Ligation Kit (Boehringer Mannheim). Optionally, the vector was gel purified without being dephosphorylated and used within the ligation reaction. The ligations for each of the chains were for performed at room temperature for 15 minutes. The new plasmid constructs were transformed into BL21 Gold (DE3) competent cells according to the Novagen protocol (Novagen, Madison, Wis.). Transformed bacteria were grown at 37° C. on LB/Kanamycin plates and individual colonies were picked and grown overnight at 37° C. in 5 ml LB broth containing 50 μg/ml kanamycin. An aliquot of each plasmid DNA was digested with the endonucleases NdeI and EcoRI and loaded onto a 1.8% agarose gel to verify the presence of the proinsulin cDNA insert. The sequence of each individual chain was verified by automated DNA sequencing (Molecular Genetics Instrumentation Facility University of Georgia, Athens, Ga.).

Expression ofA-, B-, and C-Chain

Two ml of the overnight culture were added to inoculate 500 ml of LB/kanamycin broth. The culture was grown in a shaking incubator at 37° C. until OD₆₀₀ 0.6-0.8.

Protein expression was induced by adding isopropyl-β-D-thiogalactopyranoside (IPTG) to a final concentration of 0.5 mM. The cultures were grown for an additional 1 hour. Pre-and post IPTG protein expression was analyzed using sodium dodecyl sulfate-polyacrylamide gel electrophoresis (SDS-PAGE).

The purification of each of the single chains is performed using chelating sepharose fast flow (Amersham, Piscataway, N.J.). Thus far, the C chain has been purified using this method. The proteins are eluted using a 0-1M imidazole gradient. They are then further purified using HPLC (see Example I). The proteins are then incubated with factor Xa at 37° C. for 30 minutes using 0.5 U per 10 μg of protein to remove the N-terminal methionine (Novagen, Madison, Wis.). Cleavage is monitored with SDS polyacrylamide gel electrophoresis. Factor Xa is removed during the protein purification step using RP-HPLC.

### Example V

Monoclonal and Polyclonal Antibodies Against Feline Proinsulin and Constituent A-, B-, and C-Chains

Immunogens are made by first incubating each of the polypeptides (feline proinsulin, insulin, A-chain, B-chain and C-chain) with SATA (Pierce Chemical Company, Rockford, Ill.) which introduces protected sulfhydryl groups to the proteins. The proteins are purified by gel filtration as described in the SATA protocol then cross-linked to Imject maleimide-activated mcKLH (keyhole limpet hemocyanin) with hydroxylamine hydrochloride as described in the protocol (Pierce, Rockford, Ill.) to form KLH-conjugated peptides.

To make monoclonal antibodies, mice are immunized by subcutaneous injections of either the KLH conjugated feline proinsulin, insulin, or C-peptide emulsified with Freund's complete adjuvant approximately six weeks prior to fusion, and with incomplete Freund's adjuvant two weeks prior to fusion, and again three days prior to fusion (Monoclonal Antibody facility of the University of Georgia). Spleen cells from the immunized mice showing a response to the immunogenic peptide are fused with myeloma cells to create hybridomas, and hybridomas producing antibody specific to proinsulin, insulin, or C-peptide are propagated, characterized and retained for use, for example, in immunoassays.

To make polyclonal antibodies the KLH-conjugated peptides emulsified with complete Freund's adjuvant are injected into rabbits. A pre-immune blood sample is analyzed prior to the initial injection (day 1). On day 21 a boost injection is administered (incomplete Freund's adjuvant), with subsequent injections given as described for the monoclonal antibodies.

The purified antigen are coated on polyvinyl chloride ELISA plates and, for monoclonal antibody screening, hybridoma media is incubated for 3 hours, removed, the plates washed and specific antigen-binding IgG detected by horseradish peroxidase-linked anti-mouse IgG with UV detection of peroxidase substrate product. Antigen-coated wells are compared with blanks consisting of coating buffer only. For polyclonal antibodies prepared in rabbits, titers are assessed by serial dilutions of serum from immunized rabbits. Once promising monoclonal antibodies are identified, they are used as capture antibodies to bind the antigen in solution (such as animal serum) for a sandwich ELISA with either a second monoclonal antibody or a polyclonal IgG linked to horseradish peroxidase (HRP) to quantify the captured antigen.

### Example VI

Method for Producing Monoclonal Antibodies

Monoclonal antibodies may be produced by emulsify antigen in Complete Freund's Adjuvant. The emulsion may then be used to immunize Balb/c mice (about 50-100 μg antigen per mouse given intraperitoneally). Three mice are boosted with an emulsion of antigen-Incomplete Freund's Adjuvant twice at about 21 day intervals (about 50-100 μg antigen each, given intraperitoneally). About 7 days after the second booster, an antigen-capture ELISA may be run to determine the response of the mice to the antigen. The ELISA is performed by using the antigen to coat wells of microtiter plates. After overnight incubation, coated plates are washed thoroughly, and nonspecific binding sites are blocked using 5% sucrose, 2% BSA in borate saline, pH 8.5. After incubation, plates are thoroughly washed. The primary antibody, i.e. antibody contained in the sera from the immunized mice, is diluted and added to the microtiter plate wells. Following additional washes, a goat anti-mouse IgG- and IgM- alkaline phosphatase conjugate is added to the wells. After incubation and thorough washing, the substrate for the phosphatase, TMB (3,3′,5,5′-trimethyl-benzidine; Sigma T-8665), is added to the wells.

Plates are incubated for about 10-15 minutes. Sulfuric Acid (0.1 M) is added to each well to stop the enzymatic cleavage of the substrate. Subsequently, changes in absorbance of the plate's contents are read at 405 nm with a microplate spectrophotometer as an indication of mouse response to antigen. With the identification of a positive antibody, production of monoclonal antibodies can proceed. If a positive antibody is not identified, more boosters may be used, or techniques to increase the immunogenicity of the polypeptide can be implemented as stated above.

Responding mice are given a final booster consisting of about 5-100 μg, preferably 25-50 μg of antigen, preferably without adjuvant, administered intravenously. Three to five days after final boosting, spleens and sera are harvested from all responding mice, and sera is retained for use in later screening procedures. Spleen cells are harvested by perfusion of the spleen with a syringe. Spleen cells are collected, washed, counted and the viability determined via a viability assay. Spleen and SP2/0 myeloma cells (ATCC, Rockville, Md.) are screened for HAT sensitivity and absence of bacterial contamination. The screening involves exposing the cells to a hypoxanthine, aminopterin, and thymidine selection (HAT) medium in which hybridomas survive but not lymphocytes or myeloma cells). The cells are combined, the suspension pelleted by centrifugation, and the cells fused using polyethylene glycol solution. The “fused” cells are resuspended in HT medium (RPMI supplemented with 20 % fetal bovine serum (FBS), 100 units of penicillin per ml, 0.1 mg of streptomycin per ml, 100 μMhypoxanthine, 16 uM thymidine, 50 μM 2-mercaptoethanol and 30 % myeloma-conditioned medium) and distributed into the wells of microtiter plates. Following overnight incubation at 37° C. in 5% CO₂, HAT selection medium (HT plus 0.4 μM aminopterin) is added to each well and the cells fed according to accepted procedures known in the art. In approximately 10 days, medium from wells containing visible cell growth are screened for specific antibody production by ELISA. Only wells containing hybridomas making antibody with specificity to the antigen are retained. The ELISA is performed as described above, except that the primary antibody added is contained in the hybridoma supernatants. Appropriate controls are included in each step.

This process generates several hybridomas producing monoclonal antibodies to the feline proinsulin, insulin or constituent peptide antigen. Hybridoma cells from wells testing positive for the desired antibodies are cloned by limiting dilution and re-screened for antibody production using ELISA. Cells from positive wells are subcloned to ensure their monoclonal nature. The most reactive lines are then expanded in cell culture and samples are frozen in 90% FBS-10% dimethylsulfoxide. Monoclonal antibodies can be characterized using a commercial isotyping kit (BioRad Isotyping Panel, Oakland, Calif.) and partially purified with ammonium sulfate precipitation followed by dialysis. Further purification can be performed using protein-A affinity chromatography. The antibodies, both monoclonal and polyclonal, that are directed against feline proinsulin and C-peptide may be used in diagnostic and therapeutic applications.

### Example VII

Additional Methods to Produce Monoclonal and Polyclonal Antibodies Against Feline Proinsulin and Constituent A-, B- and C-Chains

Immunogens are made by first incubating each of the polypeptides (feline A-chain, B-chain, and C-chain) with SATA (Pierce Chemical Company, Rockford, Ill.) which introduces protected sulfhydryl groups to the proteins. The proteins are purified by gel filtration as described in the SATA protocol then cross-linked to Imject maleimide- activated mcKLH (keyhole limpet hemocyanin) with hydroxylamine hydrochloride as described in the protocol (Pierce, Rockford, Ill.) to form KLH-conjugated peptides. For screening purposes, feline A-chain, B-chain, and C-chain will be conjugated to bovine serum albumin (BSA) using EDC (Pierce, Rockford, Ill.). This is necessary due to the small size of the feline peptides and their inability to efficiently bind to polyvinyl chloride plates.

Feline proinsulin and C-peptide immunogens are also prepared by glutaraldehyde aggregation of the proteins. Feline A-chain, B-chain may be prepared using glutaraldehyde aggregation as well.

To make monoclonal antibodies, mice are immunized by subcutaneous injections of one of the KLH conjugated peptides or the glutaraldehyde-aggregated peptides emulsified with Freund's complete adjuvant. Approximately six weeks prior to fusion, and with incomplete Freund's adjuvant two weeks prior to fusion, and again three days prior to fusion (Monoclonal Antibody facility of the University of Georgia). Spleen cells from the immunized mice showing a response to the immunogenic peptide are fused with myeloma cells to create hybridomas, and hybridomas producing antibody specific to proinsulin, insulin, or C-peptide are propagated, characterized and retained for use, for example, in immunoassays.

To make polyclonal antibodies the KLH-conjugated or glutaraldehyde aggregated peptides emulsified with complete Freund's adjuvant are injected into rabbits. A pre-immune blood sample is analyzed prior to the initial injection (day 1). On day 21 a boost injection is administered (incomplete Freund's adjuvant), with subsequent injections given as described for the monoclonal antibodies.

The purified antigen are coated on polyvinyl chloride ELISA plates and, for monoclonal antibody screening, hybridoma media is incubated for 3 hours, removed, the plates washed and specific antigen-binding IgG detected by horseradish peroxidase-linked anti-mouse IgG with UV detection of peroxidase substrate product. Antigen-coated wells are compared with blanks consisting of coating buffer or coating buffer with 1 μg/well BSA when screening antigen is conjugated to BSA using EDC. For polyclonal antibodies prepared in rabbits, titers are assessed by serial dilutions of serum from immunized rabbits. Once promising monoclonal antibodies are identified, they are used as capture antibodies to bind the antigen in solution (such as animal serum) for a sandwich ELISA with either a second monoclonal antibody or a polyclonal IgG linked to HRP to quantify the captured antigen.

### Example VIII

Production of Insulin and C-Peptide from Proinsulin

Purification, reduction and refolding of feline proinsulin expressed in pET 21b Proinsulin expressed in pET 21b was purified as outlined in the pET system manual (Novagen, Madison, Wis.) using the BugBuster/benzonase inclusion body purification protocol. The resulting pellet was resuspended in 5 mi/g (original weight of bacteria) 70% formic acid and incubated for 10-15 minutes. The sample was centrifuged at 10,000 g for 10 minutes at 20° C. The supernatant was applied to a Bio-Gel P-2 equilibrated with 200 mM Tris/HCl, 8M urea/ 5mM EDTA pH 8.7. The elution of protein was monitored at 280nm at a flow rate of 1.0 ml/minute and all peaks were collected. Protein concentration of the peaks was determined using the Bradford protein assay. The protein peak is the first peak in the elution profile.

The protein was further processed according to the method described by Mackin and Choquette (R. Mackin et al., Protein Expr. Purific., 27:210-219 (2003)). The purification involves reduction of the protein with dithiothreitol (60 mM final concentration). Argon was bubbled into the solution, the tube was filled with argon and the cap tightened immediately. The sample was incubated at 50° C. for 30 minutes, then cooled to room temperature and applied to the same Bio-Gel P-2 column which was equilibrated with 50 mM glycine/NaOH, 1 mM EDTA, pH 10.5, at a flow rate of 1 ml/minute. The protein peak was collected.

The amount of proinsulin was determined using the Bradford protein assay and the peptide was diluted to a final protein concentration of 100 μg/ml using fresh 50 mM glycine/NaOH, pH 10.5, 1 mM EDTA buffer. Both reduced and oxidized glutathione were added to a final concentration of 1 mM each. The sample was briefly bubbled with argon, the tube filled with argon and immediately sealed. The sealed sample was incubated at 4° C. overnight.

Folded proinsulin was purified using a reverse phase column (Vydac 259VHP822 prep column 22 mm×250 mm; VYDAC/The Separations Group, Inc, Hesperia, Calif.). The column was equilibrated with 75% 0.1% TFA in H₂O/25% 0.1% TFA in 80% acetonitrile. The sample was prepared for injection to contain a final solution of 10% acetonitrile. The filtered sample was injected and eluted using a linear gradient of 0.1% trifluoroacetic acid (TFA) (A) and 0.1% TFA in 20% H₂O/80% acetonitrile (B) increasing B from 25% to 32% in 4 minutes and from 32-42% in 30 minutes. Human recombinant proinsulin (Eli Lilly, Indianapolis, Ind.) was used as a standard. The molecular weight of purified feline proinsulin was determined by mass-spectroscopy (Proteomics Facility, University of Georgia, Athens, Ga.). The purity of the feline proinsulin was determined using RP-HPLC on a Jupiter C4 4.6 mm×250 mm column under similar conditions as purification (OOF-4167-E0, Phenomenex, Torrance, Calif.).

The purified proinsulin was converted to insulin and C-peptide using the method as described in Example I (Kemmler et al., J. Biol. Chem., 246: 6780-6791 (1971)).

### Example IX

Purification, Reduction and Refolding Offeline Proinsulin Expressed in pET28

Proinsulin expressed in pET 28 was purified as outlined in the pET system manual (Novagen, Madison, Wis.). Cells were lysed using the BugBuster/bensonase inclusion body protocol (as described above in Example I for proinsulin expressed in pET21). The protein was reduced as per the previous protocol and the resultant protein was purified by RP-HPLC as previously described for folded proinsulin from pET 21b. The fusion tag was enzymatically cleaved from the purified proinsulin by incubation with Factor Xa at 30° C. for 30 minutes. The Factor Xa was removed using Xarrest agarose (Novagen, Madison, Wis.). This cleaved protein represents the native sequence of feline proinsulin. This proinsulin was applied to a Bio-Gel P-2 column equilibrated in 50 mM glycine/NaOH, 1 mM EDTA, pH 10.5 at 1 mL/minute. The protein peak was collected and diluted to 100μg/ml with fresh glycine buffer. Oxidized and reduced glutathione was added to a concentration of 1 mM each. The solution was de-gassed using argon and incubated overnight at 4° C. to fold the proinsulin. The folded proinsulin was then purified using RP-HPLC as previously described for proinsulin from pET 21b. The collected folded protein was assayed for protein content, analyzed with SDS page electrophoresis and by MALDI-TOF mass spectrometry for molecular weight analysis. The resultant purified protein can be converted to insulin and C-peptide as outlined in Example I (method described by Kemmler et al. J. Biol. Chem. 246: 6780-6791, 1971).

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. An isolated C-chain peptide consisting essentially of an amino acid sequence

| | |
|---|---|
| EAED | (SEQ ID NO: 9) |
| LQGKDAELGEAPGAGGLQPSALEAPLQ. | |

2. An isolated C-chain peptide comprising an amino acid sequence having at least 80% identity to an amino acid sequence

| | |
|---|---|
| EAEDLQGKDAELGEAPGAGGLQPS | (SEQ ID NO: 9) |
| ALEAPLQ. | |

3. The C-chain peptide of claim 2 which improves renal function in diabetic mammals when administered with insulin.

4. The C-chain peptide of claim 2 which reduces duration-dependent hippocampal apoptosis resulting from type-1 diabetes, promotes neurite proliferation, promotes neurite outgrowth, promotes autophosphorylation of an insulin receptor, stimulates phosphoinositide 3-kinase, stimulates p38 mitogen-activated protein kinase, promotes expression of nuclear factor-kappaB, promotes nuclear tranlocation of nuclear factor-kappaB, promotes expression of Bcl2, stimulates Na(+),K(+)-ATPase, stimulates nitric oxide synthase, raises intracellular Ca+2 concentration, reduces nerve dysfunction in patients with diabetic neuropathy, or reduces c-jun N-terminal kinase phosphorylation in diabetic mammals when administered with insulin.

5. An isolated subunit of a C-chain peptide having an amino acid sequence

| | |
|---|---|
| ELGEAP GAG, or | (SEQ ID NO: 34) |
| EAPLQ. | (SEQ ID NO: 35) |

6. The subunit peptide of claim 5, wherein the subunit stimulates Na(+),K(+)-ATPase activity.

7. An isolated polypeptide comprising a subunit of a C-chain peptide having an amino acid sequence E L G E A P G A G (SEQ ID NO:34), or E A P L Q (SEQ ID NO:35) that stimulates Na(+),K(+)-ATPase.

8. An isolated C-chain peptide comprising an amino acid sequence

| | |
|---|---|
| EAEDLQGKD | |
| AELGEAPGAGGLQPSALEAPLQ. | (SEQ ID NO: 9) |

9. An isolated proinsulin polypeptide comprising SEQ ID NO:9.

10. An isolated proinsulin polypeptide comprising an amino acid sequence having at least 80% identity to SEQ ID NO:9.

11. An isolated proinsulin polypeptide consisting essentially of SEQ ID NO:1.

12. An isolated proinsulin polypeptide comprising an amino acid sequence having at least 85% identity to SEQ ID NO:1.

13. The proinsulin polypeptide of claim 12 which reduces blood glucose levels when administered to a mammal.

14. An isolated proinsulin polypeptide comprising SEQ ID NO:1.

15. An isolated proinsulin polypeptide comprising SEQ ID NOs: 5, 9, and 13.

16. The isolated proinsulin polypeptide of claim 15, further comprising at least one cleavable linker.

17. A peptidomimetic of SEQ ID NO. 1, 5, 9, or 13.

18. An isolated polynucleotide comprising a coding region that encodes SEQ ID NO:9.

19. An isolated polynucleotide comprising a coding region that encodes SEQ ID NO:1.

20. An expression cassette comprising a regulatory sequence operably linked to a polynucleotide that encodes SEQ ID NO:9.

21. The expression cassette of claim 20, wherein the regulatory sequence is a promoter, operator, intron, repressor binding site, enhancer, or any combination thereof.

22. A vector comprising a polynucleotide that encodes SEQ ID NO:9.

23. An antibody that binds to at least one polypeptide selected from the group consisting of feline A-chain peptide; feline B-chain peptide; feline C-chain peptide, feline insulin and feline proinsulin.

24. The antibody of claim 23 that does not bind to human, porcine or bovine insulin or a constituent peptide thereof.

25. A peptide aptamer that binds to at least one polypeptide selected from the group consisting of feline A-chain peptide, feline B-chain peptide, feline C-chain peptide, feline insulin, and feline proinsulin.

26. A pharmaceutical composition comprising a pharmaceutically acceptable carrier and at least one component selected from the group consisting of a feline proinsulin, insulin, subunit, constituent peptide thereof, analog or derivative thereof, peptidomimetic thereof, or antibody or peptide aptamer that specifically binds thereto.

27. The pharmaceutical composition according to claim 26 that is formulated as a single unit dosage.

28. A method for making a feline proinsulin, insulin, a subunit, or constituent peptide thereof comprising:
providing a host cell comprising a nucleotide sequence that encodes the proinsulin, insulin, subunit, or constituent peptide thereof; and
expressing the proinsulin, insulin, subunit, or constituent peptide thereof in the host cell.

29. The method of claim 28 further comprising purifying the expressed proinsulin, insulin, subunit, or constituent peptide.

30. A method for predicting or diagnosing diabetes in a cat comprising determining the ratio of insulin to proinsulin in a biological fluid obtained from the cat.

31. A method for predicting or diagnosing diabetes in a cat comprising determining the ratio of insulin to C-peptide in a biological fluid obtained from the cat.

32. A method to determine if a cat is predisposed to develop neuropathy, retinopathy or nephropathy comprising determining if C-peptide concentration in a biological fluid obtained from the cat is less than a predetermined amount.

33. A method for treating a mammal suspected of having diabetes comprising administering to the mammal at least one polypeptide selected from the group consisting of a feline proinsulin, feline insulin, a feline A-chain peptide, a feline B-chain peptide, a feline C-chain peptide, a constituent polypeptide, a subunit, a peptidomimetic, an analog, or any combination thereof.

34. A method for treating a mammal suspected of having diabetes comprising administering to the mammal at least one polypeptide selected from the group consisting of a feline proinsulin, a feline insulin, a feline A- chain peptide, and a feline C-chain peptide, wherein the feline proinsulin, feline insulin, feline A- chain peptide or feline C-chain peptide has been modified to yield an proinsulin, insulin, A-chain peptide or C-chain peptide analogous to insulin lispro, insulin aspart, insulin glargine or detemir insulin.

35. A method to identify an antiproliferative factor comprising:
incubating neuroblastoma test cells with feline C-peptide, insulin, and a candidate antiproliferative factor; and
comparing proliferation of the test cells to proliferation of neuroblastoma control cells that were incubated with feline C-peptide and insulin.

36. A method to identify an antiproliferative factor comprising:
incubating neuroblastoma test cells with feline C-peptide, insulin, and a candidate antiproliferative factor; and
comparing autophosphorylation of insulin receptors within the test cells to autophosphorylation of insulin receptors within control cells that were incubated with feline C-peptide and insulin.

37. A method to reduce or ameliorate a diabetes associated disorder in a mammal comprising administering an effective amount of a feline C-peptide, a peptidomimetic of feline C-peptide, a subunit of a feline C-peptide, an analog, a peptidomimetic of a subunit of a feline C-peptide, or any combination thereof to the mammal in need of such treatment.

38. The method of claim 37 further comprising administering insulin to the mammal.

39. The method of claim 37, wherein the mammal is a cat.

40. A kit comprising packaging material and a first antibody that specifically binds to a feline C-peptide, and a second antibody that specifically binds to feline insulin.

41. The kit of claim 40, wherein at least one of the first antibody or the second antibody is coupled to a detectable marker.

42. A kit comprising packaging material and a first antibody that specifically binds to a C-peptide chain within feline proinsulin, and a second antibody that specifically binds to feline insulin.

43. The kit of claim 42, wherein at least one of the first antibody or the second antibody is coupled to a detectable marker.


SEQUENCE LISTING (35 sequences)

SEQ ID NO: 1 - protein, 86 aa, Felis catus
FVNQHLCGSHLVEALYLVCGERGFFYTPKARREAEDLQGKDAELGEAPGAGGLQPSALEAPLQKRGIVEQCCASVCSLYQLEHYCN

SEQ ID NO: 2 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 3 - protein, 84 aa, Sus scrofa
FVNQHLCGSHLVEALYLVCGERGFFYTPKARREAENPQAGAVELGGGLGGLQALALEGPPQKRGIVEQCCTSICSLYQLENYCN

SEQ ID NO: 4 - protein, 81 aa, Bos taurus
FVNQHLCGSHLVEALYLVCGERGFFYTPKARREVEGPQVGALELAGGPGAGGLEGPPQKRGIVEQCCASVCSLYQLENYCN

SEQ ID NO: 5 - protein, 30 aa, Felis catus
FVNQHLCGSHLVEALYLVCGERGFFYTPKA

SEQ ID NO: 6 - protein, 30 aa, Homo sapiens
FVNQHLCGSHLVEALYLVCGERGFFYTPKT

SEQ ID NO: 7 - protein, 30 aa, Sus scrofa; identical to SEQ ID NO: 5

SEQ ID NO: 8 - protein, 30 aa, Bos taurus; identical to SEQ ID NO: 5

SEQ ID NO: 9 - protein, 31 aa, Felis catus
EAEDLQGKDAELGEAPGAGGLQPSALEAPLQ

SEQ ID NO: 10 - protein, 31 aa, Homo sapiens
EAEDLQVGQVELGGGPGAGSLQPLALEGSLQ

SEQ ID NO: 11 - protein, 29 aa, Sus scrofa
EAENPQAGAVELGGGLGGLQALALEGPPQ

SEQ ID NO: 12 - protein, 26 aa, Bos taurus
EVEGPQVGALELAGGPGAGGLEGPPQ

SEQ ID NO: 13 - protein, 21 aa, Felis catus
GIVEQCCASVCSLYQLEHYCN

SEQ ID NO: 14 - protein, 21 aa, Homo sapiens
GIVEQCCTSICSLYQLENYCN

SEQ ID NO: 15 - protein, 21 aa, Sus scrofa
GIVEQCCTSICSLYQLENYCN

SEQ ID NO: 16 - protein, 21 aa, Bos taurus
GIVEQCCASVCSLYQLENYCN

SEQ ID NO: 17 - DNA, 40 bp, Artificial Sequence
  Description of Artificial Sequence Primer
gcgaattctgcaggatccaaactttttttttttttttttt

SEQ ID NO: 18 - DNA, 27 bp, Artificial Sequence
  Description of Artificial Sequence Primer
cctgccccgacccgagccttcgtcaac

SEQ ID NO: 19 - DNA, 90 bp, Felis catus
ttcgtcaaccagcacctgtgcggctcccacctggtggaggcgctgtacctggtgtgcggggagcgcggcttcttctacacgcccaaggcc

SEQ ID NO: 20 - DNA, 105 bp, Felis catus
cgccgggaggcggaggacctccaggggaaggacgcggagctgggggaggcgcctggcgccggcggcctgcagccctcggccctggaggcgcccctgcagaagcgg

SEQ ID NO: 21 - DNA, 63 bp, Felis catus
ggcatcgtggagcaatgctgtgccagcgtctgctcgctgtaccagctggagcattactgcaac

SEQ ID NO: 22 - DNA, 258 bp, Felis catus
ttcgtcaaccagcacctgtgcggctcccacctggtggaggcgctgtacctggtgtgcggggagcgcggcttcttctacacgcccaaggcccgccgggaggcggaggacctccaggggaaggacgcggagctgggggaggcgcctggcgccggcggcctgcagccctcggccctggaggcgcccctgcagaagcggggcatcgtggagcaatgctgtgccagcgtctgctcgctgtaccagctggagcattactgcaac

SEQ ID NO: 23 - DNA, 27 bp, Artificial Sequence
  Description of Artificial Sequence Primer
ctccatatgttcgttaaccagcacctg

SEQ ID NO: 24 - DNA, 30 bp, Artificial Sequence
  Description of Artificial Sequence Primer
gcgggatccctagttgcagtagtgttccag

SEQ ID NO: 25 - DNA, 45 bp, Artificial Sequence
  Description of Artificial Sequence Primer
ggatcccatatgatcgaaggtcgtttcgtcaaccagcacctgtgc

SEQ ID NO: 26 - DNA, 32 bp, Artificial Sequence
  Description of Artificial Sequence Primer
cggaattcctagttgcagtagtgttccagctg

SEQ ID NO: 27 - DNA, 47 bp, Artificial Sequence
  Description of Artificial Sequence Primer
ggatcccatatgatcgaaggtcgtggtatcgttgaacagtgctgcgc

SEQ ID NO: 28 - DNA, 34 bp, Artificial Sequence
  Description of Artificial Sequence Primer
cggaattctacgctttcggggtgtagaagaaacc

SEQ ID NO: 29 - DNA, 45 bp, Artificial Sequence
  Description of Artificial Sequence Primer
ggatcccatatgatcgaaggtcgtcgtcgtgaagcggaagacctg

SEQ ID NO: 30 - DNA, 32 bp, Artificial Sequence
  Description of Artificial Sequence Primer
cggaattcctaacgtttctgcagcggcgcttc

SEQ ID NO: 31 - DNA, 45 bp, Artificial Sequence
  Description of Artificial Sequence Primer
ggatcccatatgatcgaaggtcgtgaagcggaagacctgcagggt

SEQ ID NO: 32 - DNA, 38 bp, Artificial Sequence
  Description of Artificial Sequence Primer
cggaattcctactgcagcggcgcttccagcgcagacgg

SEQ ID NO: 33 - DNA, 258 bp, Artificial Sequence
  Description of Artificial Sequence codon-optimized proinsulin
ttcgttaaccagcacctgtgcggttctcacctggttgaagcgctgtacctggtttgcggtgaacgtggtttcttctacaccccgaaagcgcgtcgtgaagcggaagacctgcagggtaaagacgcggaactgggtgaagcgccgggtgcgggtggtctgcagccgtctgcgctggaagcgccgctgcagaaacgtggtatcgttgaacagtgctgcgcgtctgtttgctctctgtaccagctggaacactactgcaac

SEQ ID NO: 34 - protein, 9 aa, Felis catus
ELGEAPGAG

SEQ ID NO: 35 - protein, 5 aa, Felis catus
EAPLQ